(19) United States
(12) Patent Application Publication
(10) Pub. No.: US 2012/0088246 A1
(43) Pub. Date: Apr. 12, 2012

(54) REAL TIME PCR DETECTION OF SINGLE NUCLEOTIDE POLYMORPHISMS

(75) Inventors: Jason OPDYKE, Silver Spring, MD (US); John HARVEY, Elkridge, MD (US)

(73) Assignee: SAMSUNG TECHWIN CO., LTD., CHANGWON-CITY (KR)

(21) Appl. No.: 13/158,593

(22) Filed: Jun. 13, 2011

Related U.S. Application Data
(60) Provisional application No. 61/390,701, filed on Oct. 7, 2010.

(51) Int. Cl. C12Q 1/68

(52) U.S. Cl. 435/6.12

(57) ABSTRACT

Disclosed are methods and kits for the detection of a polymorphism during real-time PCR. Real-time PCR amplification of a target nucleic acid sequence is performed using PCR primer primers that anneal to sequences flanking a single nucleotide polymorphism (SNP) of interest. The real-time PCR reaction includes a labeled probe comprising a RNA sequence that is designed to anneal to DNA sequences at the location of the SNP. An RNA:DNA heteroduplex can then form between the SNP in the PCR fragment and the probe's RNA sequences that are complementary to the SNP. RNase H cleavage of the RNA sequence in the RNA:DNA heteroduplex results in increase in intensity of the signal generated from the label that is indicative of the presence of an SNP in the target nucleic acid.

### CROSS-REFERENCE TO RELATED APPLICATIONS

This application claims priority from U.S. Provisional Patent Application No. 61/390,701, filed on Oct. 7, 2010, the contents of which are hereby incorporated by reference in their entirety.

### FIELD

The disclosure describes real-time PCR detection of Single Nucleotide Polymorphisms (SNPs) using a SNP-specific CataCleave™ probe.

### BACKGROUND

Completion of the Human Genome Project (HGP) has paved the way to a greater understanding of genetic diversity within a given human population and how that diversity relates to the onset or predisposition to genetic disease. For example, single nucleotide differences between individuals, called Single Nucleotide Polymorphisms (SNPs), can be responsible for dramatic differences in phenotype that in some circumstances may predict who will be afflicted with certain diseases or who will respond to a particular treatment for those diseases. The realm of pharmacogenomics is fast approaching where medical treatment is tailored to a patient's genetic make-up. Rapid and reliable detection of genetic mutations in a patient's DNA can guide a physician's clinical diagnosis and choice of medical treatment. This is particularly true in oncology where the presence of discrete mutations within the coding regions of oncogenes can be powerful predictors of cancer susceptibility and prognosis.

For example, genetic testing for the presence of harmful mutations within the BRCA tumor suppressor genes are is now routine in most medical practices. Harmful BRCA-1 mutations can greatly increase a woman's risk of developing breast and/or ovarian cancer at an early age (before menopause) as well as the risk of developing cervical, uterine, pancreatic, and colon cancer. Harmful BRCA2 mutations may additionally increase the risk of pancreatic cancer, stomach cancer, gallbladder and bile duct cancer, and melanoma. Mutations in several other genes, including TP53, PTEN, STK11/LKB1, CDH1, CHEK2, ATM, MLH1, and MSH2, have been associated with hereditary breast and/or ovarian tumors.

With the advent of nucleic acid amplification, as little as a single molecule of any DNA sequence can be copied a sufficient number of times to permit SNP sequence analysis. SNPs may be detected by a variety of techniques, such as DNA sequencing, fluorescent probe detection, mass spectrometry or DNA microarray hybridization (e.g., U.S. Pat. Nos. 5,885,775; 6,368,799). Many of these procedures remain inadequate however for high throughput applications because of either overall poor sensitivity, cost, time expenditure or the need for post-PCR processing. Existing methods of SNP detection also have an unacceptably high level of false positive and/or false negative results.

For the foregoing reasons, there is an unmet need in the art for the accurate real time detection of SNPs concurrent with DNA amplification.

### SUMMARY

Methods and kits are described for the rapid detection of SNPs during real time PCR using a specific probe which is comprised of DNA and RNA sequences. The procedure promises to facilitate the high throughput detection of one or more SNPs on a single PCR fragment in a cost effective and reliable manner.

In one embodiment, there is disclosed a method for the real-time detection of a polymorphism in a target DNA, comprising the steps of providing a sample to be tested for the presence of a target DNA having a polymorphism, providing a pair of amplification primers that can anneal to the target DNA, wherein a first amplification primer anneals upstream of the location of the polymorphism and the second amplification primer anneals downstream of the location of the polymorphism, providing a probe comprising a detectable label and DNA and RNA nucleic acid sequences, wherein the probe's RNA nucleic acid sequences are entirely complementary to a selected region of the target DNA sequence comprising the polymorphism and the probe's DNA nucleic acid sequences are substantially complementary to DNA sequences adjacent to the selected region of the target DNA sequence, amplifying a PCR fragment between the first and second amplification primers in the presence of an amplifying polymerase activity, amplification buffer; an RNase H activity and the probe under conditions where the RNA sequences within the probe can form a RNA:DNA heteroduplex with the complementary DNA sequences in the PCR fragment comprising the polymorphism, and detecting a real-time increase in the emission of a signal from the label on the probe, wherein the increase in signal indicates the presence of the polymorphism in the target DNA.

In one aspect, the real-time increase in the emission of the signal from the label on the probe results from the RNase H cleavage of the probe's RNA sequences in the RNA:DNA heteroduplex.

In another embodiment, there is disclosed a method for the real-time detection of a polymorphism in a target DNA, comprising steps of providing a sample to be tested for the presence of a target DNA having a polymorphism, providing a pair of amplification primers that can anneal to the target DNA, wherein a first amplification primer anneals upstream of the location of the polymorphism and the second amplification primer anneals downstream of the location of the polymorphism, providing a probe comprising a detectable label and DNA and RNA nucleic acid sequences, wherein the probe's RNA nucleic acid sequences are entirely complementary to a selected region of the target DNA sequence comprising a wild type DNA sequence at the location of the polymorphism and the probe's DNA nucleic acid sequences are substantially complementary to DNA sequences adjacent to the selected region of the target DNA sequence, amplifying a PCR fragment between the first and second amplification primers in the presence of an amplifying polymerase activity, amplification buffer; an RNase H activity and the probe under conditions where the RNA sequences within the probe can form a RNA:DNA heteroduplex with the complementary DNA sequences in the PCR fragment comprising the polymorphism, and detecting a real-time decrease in the emission of a signal from the label on the probe, wherein the decrease in signal indicates the presence of the polymorphism in the target DNA.

In another embodiment, there is disclosed a method for the real-time detection of a polymorphisms in a RNA target, comprising the steps of providing an RNA target, providing a pair of amplification primers that can anneal to a cDNA of the RNA target, wherein a first amplification primer anneals upstream of the location of the polymorphic sequence and the second amplification primer anneals downstream of the location of the polymorphic sequence; providing a probe comprising a detectable label and DNA and RNA nucleic acid sequences that are substantially complimentary to the cDNA of the RNA target, wherein the RNA nucleic acid sequence of the probe comprises a sequence that is entirely complimentary to the corresponding cDNA sequence at the location of the suspected SNP sequence; amplifying a reverse transcriptase-PCR fragment between the first and second amplification primers in the presence of a reverse transcriptase activity, an amplifying polymerase activity, a reverse transcriptase-PCR buffer, a site-specific RNase H activity and the probe and under conditions where the RNA sequences within the probe can form a RNA: DNA heteroduplex with complimentary sequences in the RT-PCR DNA fragment; and detecting a real-time increase in the emission of a signal from the label on the probe, wherein the increase in signal indicates the presence of the polymorphism in the cDNA of the RNA target.

In one aspect, the real-time increase in the emission of the signal from the label on the probe results from the RNase H cleavage of the probe's RNA sequences in the RNA:DNA heteroduplex.

In another embodiment, there is disclosed a method for the real-time detection of a polymorphisms in a RNA target, comprising the steps of providing a sample to be tested for the RNA target having a polymorphism, providing a pair of amplification primers that can anneal to a cDNA of the RNA target, wherein a first amplification primer anneals upstream of the location of the polymorphism and the second amplification primer anneals downstream of the location of the polymorphism, providing a probe comprising a detectable label and DNA and RNA nucleic acid sequences, wherein the probe's RNA nucleic acid sequences are entirely complementary to a selected region of the cDNA comprising a wild type DNA sequence at the location of the polymorphism and the probe's DNA nucleic acid sequences are substantially complementary to DNA sequences adjacent to the selected region of the cDNA, amplifying a reverse transcriptase-PCR fragment between the first and second amplification primers in the presence of a reverse transcriptase activity, an amplifying polymerase activity, a reverse transcriptase-PCR buffer; an RNase H activity and the probe and under conditions where the RNA sequences within the probe can form a RNA:DNA heteroduplex with complementary sequences in the RT-PCR DNA fragment comprising the wild type DNA sequence at the location of the polymorphism; and detecting a real-time decrease in the emission of a signal from the label on the probe, wherein the decrease in signal indicates the presence of the polymorphism in the RNA target.

In another embodiment, there is disclosed a kit for the real-time detection of a polymorphisms in a target DNA comprising a pair of amplification primers that can anneal to a target DNA, wherein a first amplification primer anneals upstream of the location of a polymorphism and a second amplification primer anneals downstream of the location of the polymorphism, a probe comprising a detectable label and DNA and RNA nucleic acid sequences, wherein the probe's RNA nucleic acid sequences are entirely complementary to a selected region of the target DNA sequence comprising the polymorphism and the probe's DNA nucleic acid sequences are substantially complementary to DNA sequences adjacent to the selected region of the target DNA sequence and an amplifying polymerase activity, an amplification buffer; and an RNase H activity.

In another embodiment, there is disclosed a kit for the real-time detection of a polymorphism in a target DNA comprising a pair of amplification primers that can anneal to a target DNA, wherein a first amplification primer anneals upstream of the location of a polymorphism and a second amplification primer anneals downstream of the location of the polymorphism, a probe comprising a detectable label and DNA and RNA nucleic acid sequences, wherein the probe's RNA nucleic acid sequences are entirely complementary to a selected region of the target DNA sequence comprising the wild type DNA sequence at the location of the polymorphism and the probe's DNA nucleic acid sequences are substantially complementary to DNA sequences adjacent to the selected region of the target DNA sequence and an amplifying polymerase activity, an amplification buffer; and an RNase H activity.

In another embodiment, there is disclosed a kit for the real-time detection of a polymorphism in a RNA target comprising a pair of amplification primers that can anneal to a cDNA of the RNA target, wherein a first amplification primer anneals upstream of the location of a polymorphic sequence and a second amplification primer anneals downstream of the location of the polymorphic sequence, a probe comprising a detectable label and DNA and RNA nucleic acid sequences, wherein the probe's RNA nucleic acid sequences are entirely complementary to a selected region of the cDNA comprising the polymorphism and the probe's DNA nucleic acid sequences are substantially complementary to DNA sequences adjacent to the selected region of the cDNA, and a reverse transcriptase activity, an amplifying polymerase activity, reverse transcriptase-PCR buffer; and an RNase H activity.

In another embodiment, there is disclosed a kit for the real-time detection of a polymorphism in a RNA target comprising a pair of amplification primers that can anneal to a cDNA of the RNA target, wherein a first amplification primer anneals upstream of the location of a polymorphic sequence and a second amplification primer anneals downstream of the location of the polymorphic sequence, a probe comprising a detectable label and DNA and RNA nucleic acid sequences, wherein the probe's RNA nucleic acid sequences are entirely complementary to a selected region of the cDNA comprising the wild type DNA sequence at the location of the polymorphism and the probe's DNA nucleic acid sequences are substantially complementary to DNA sequences adjacent to the selected region of the cDNA, and a reverse transcriptase activity, an amplifying polymerase activity, reverse transcriptase-PCR buffer; and an RNase H activity.

The polymorphism can be a single nucleotide polymorphism (SNP).

The target DNA can be genomic DNA. The target RNA can be genomic RNA or an mRNA transcript.

The DNA and RNA sequences of the probe can be covalently linked.

The detectable label on the probe can be a fluorescent label such as a FRET pair. The PCR fragment or probe may be linked to a solid support.

The amplifying polymerase activity may be an activity of a thermostable DNA polymerase and the site-specific RNase H activity may be the activity of a thermostable RNase H or a hot start thermostable RNase H activity.

In certain embodiments, the reverse transcriptase activity and the amplifying polymerase activity are found on a same molecule

The previously described embodiments have many advantages, including the ability to detect the presence of a SNP in a target nucleic acid in real-time. The detection method is fast, accurate and suitable for high throughput applications. Convenient, user-friendly and reliable diagnostic kits are also described for the detection of SNPs at different genetic loci.

### BRIEF DESCRIPTION OF THE DRAWINGS

The skilled artisan will understand that the drawings, described below, are for illustration purposes only. The figures are not intended to limit the scope of the teachings in any way.

FIG. 1 depicts the isothermal detection of wild type target in the Salmonella invA gene. An increase in fluorescence intensity is seen for the correctly paired (wild type probe:wild type target) but not for an incorrectly paired (wild type probe:SNP containing target).

FIG. 2 depicts the isothermal detection of SNP target in the Salmonella invA gene (T to G base change). An increase in fluorescence intensity is seen for the correctly paired (SNP probe:SNP target) but not for an incorrectly paired (SNP probe:wild type target).

FIG. 3 depicts a multiplex real time detection of wild type target in the Salmonella invA gene using wild type sensing probe. In the presence of homozygous wild type target or heterozygous wild type-SNP target, an increase in fluorescence intensity is seen. An increase in fluorescence intensity is not observed in the presence of homozygous SNP target.

FIG. 4 depicts a multiplex real time detection of SNP target in the Salmonella invA gene using SNP sensing probe. In the presence of homozygous SNP target or heterozygous SNP-wild type target, an increase in fluorescence intensity is seen. An increase in fluorescence intensity is not observed in the presence of homozygous wild type target.

FIG. 5 depicts the isothermal detection of A1β casein. An increase in fluorescence intensity is seen for the correctly paired (A1 probe:A1 target) but not for an incorrectly paired (A1 probe:A2 target).

FIG. 6 depicts the isothermal detection of A2β casein. An increase in fluorescence intensity is observed with the correctly paired A2 probe:A2 target but not for an incorrectly paired A2 probe:A1 target.

FIG. 7 depicts a multiplex real time detection of A1β casein using A1 sensing probe. In the presence of homozygous A1 target or heterozygous A1-A2 target, an increase in fluorescence intensity is seen. An increase in fluorescence intensity is not observed in the presence of homozygous A2 target.

FIG. 8 depicts a multiplex real time detection of A2β casein using A2 sensing probe. In the presence of homozygous A2 target or heterozygous A1-A2 target an increase in fluorescence intensity is seen. An increase in fluorescence intensity is not observed in the presence of homozygous A1 target.

FIG. 9 is a Table describing all of the PCR primers, probes, and targets used in the figures and examples. The locations of the SNPs are underlined.

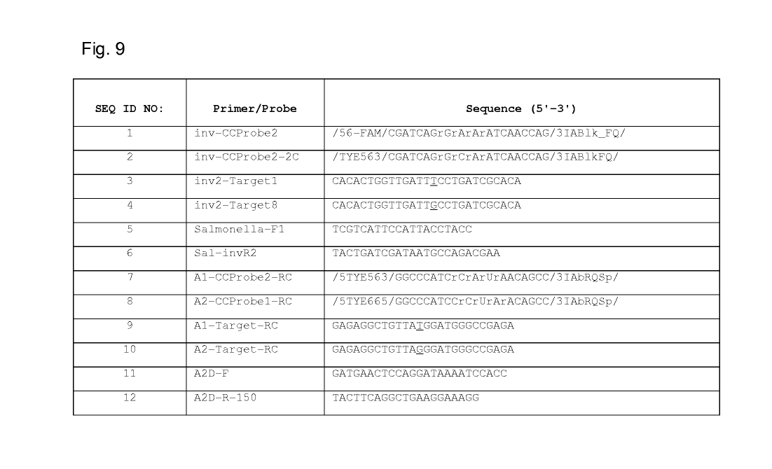

FIG. 10 depicts a sequence alignment between Pyrococcus furiosis, Pyrococcus horikoshi, Thermococcus kodakarensis, Archeoglobus profundus, Archeoglobus fulgidis, Thermococcus celer and Thermococcus litoralis RNase HII polypeptide sequences.

FIG. 11 depicts sequence alignment of Haemophilus influenzae, Thermus thermophilis, Thermus acquaticus, Salmonella enterica and Agrobacterium tumefaciens RNase HI polypeptide sequences.

### DETAILED DESCRIPTION

The practice of the invention employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

The term “polymorphism” refers to the occurrence of two or more alternative genomic sequences or alleles between or among different genomes or individuals.

The term “polymorphic” refers to the condition in which two or more variants of a specific genomic sequence are found in a population.

The term “polymorphic site” is the locus at which the variation occurs. A polymorphic site generally has at least two alleles, each occurring at a significant frequency in a selected population. A polymorphic locus may be as small as one base pair, in which case it is referred to as single nucleotide polymorphism (SNP). The first identified allelic form is arbitrarily designated as the reference, wild-type, common or major form, and other allelic forms are designated as alternative, minor, rare or variant alleles.

The term “genotype” refers to a description of the alleles of a gene contained in an individual or sample.

The term “single nucleotide polymorphism” (“SNP”) refers to a site of one nucleotide that varies between alleles. Single nucleotides may be changed (substitution), removed (deletions) or added (insertion) to a polynucleotide sequence. Insertion or deletion SNPs may cause a translational frameshift. Single nucleotide polymorphisms may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation) but if a different polypeptide sequence is produced they are nonsynonymous. A nonsynonymous change may either be missense or nonsense, where a missense change results in a different amino acid, while a nonsense change results in a premature stop codon. “Functional SNPs” are SNPs that produce alterations in gene expression or in the expression or function of a gene product, and therefore are most predictive of a possible clinical phenotype. The alterations in gene function caused by functional SNPs may include changes in the encoded polypeptide, changes in mRNA stability, binding of transcriptional and translation factors to the DNA or RNA, and the like. SNPs that are not in protein-coding regions may still have consequences for gene splicing, transcription factor binding, or the sequence of non-coding RNA.

In accordance with an embodiment, one of a skilled artisan understands that SNPs have two alternative alleles, each corresponds to a nucleotide that may exist in the chromosome. Thus, a SNP is characterized by two nucleotides out of four (A, C, G, T). An example would be that a SNP has either allele C or allele T at a given position on each chromosome. This is shown as C>T or C/T. The more commonly occurring allele is shown first (in this case, it is C) and called the major, common or wild-type allele. The alternative allele that occurs less commonly instead of the common allele (in this case, it is T) is called minor, rare or variant allele. Wild-type and variant alleles may be referred to as common and rare alleles respectively. Since humans are diploid organisms meaning that each chromosome occurs in two copies, each individual has two alleles at a SNP. These alleles may be two copies of the same allele (CC or TT) or they may be different ones (CT). The CC, CT and TT are called genotypes. Among these CC and TT are characterized by having two copies of the same allele and are called homozygous genotypes. The genotype CT has different alleles on each chromosome and is a heterozygous genotype. Individuals bearing homozygote or heterozygote genotypes are called homozygous and heterozygous, respectively.

### Selection of SNPs

An embodiment provides a novel procedure to detect one or more SNPs in any targeted nucleic acid sequence. The determination of the location of SNPs in genes of interest is greatly facilitated by reference to bioinformatics databases for SNPs. dbSNP is a SNP database from the National Center for Biotechnology Information (NCBI). SNPedia is a wiki-style database from a hybrid organization. The OMIM database describes the association between polymorphisms and, e.g., diseases in text form, while HGVbaseG2P allows users to visually interrogate the actual summary-level association data.

Invaluable information about SNPs can also be found at The International HapMap Project that seeks to genotype one informative SNP approximately every 5 kb throughout the human genome. Populations with ancestry from Nigeria, Europe, and China/Japan are being genotyped to determine the common patterns of human DNA sequence variation (haplotypes) and to make this information freely available in the public domain. The information will facilitate discovery of sequence variants that affect common disease and pharmaceutical response. Constructing the human haplotype map is a significant step towards personalized medicine.

### Selection of Primers for Genotyping

Once the genes and associated SNPs are selected, primer oligonucleotides and probes are prepared for the genotyping of a target nucleic acid sequence.

A “target DNA or “target RNA” or “target nucleic acid,” or “target nucleic acid sequence” refer to a region of nucleic acid that is to be analyzed and comprises the polymorphic site of interest. A target nucleic acid sequence serves as a template for amplification in a PCR reaction or reverse transcriptase-PCR reaction. Target nucleic acid sequences may include both naturally occurring and synthetic molecules. Exemplary target nucleic acid sequences include, but are not limited to, genomic DNA or genomic RNA.

As used herein, the term “nucleic acid” refers to an oligonucleotide or polynucleotide, wherein said oligonucleotide or polynucleotide may be modified or may comprise modified bases. Oligonucleotides are single-stranded polymers of nucleotides comprising from 2 to 60 nucleotides. Polynucleotides are polymers of nucleotides comprising two or more nucleotides. Polynucleotides may be either double-stranded DNAs, including annealed oligonucleotides wherein the second strand is an oligonucleotide with the reverse complement sequence of the first oligonucleotide, single-stranded nucleic acid polymers comprising deoxythymidine, single-stranded RNAs, double stranded RNAs or RNA/DNA heteroduplexes. Nucleic acids include, but are not limited to, genomic DNA, cDNA, hnRNA, snRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample. Nucleic acids may be composed of a single type of sugar moiety, e.g., as in the case of RNA and DNA, or mixtures of different sugar moieties, e.g., as in the case of RNA/DNA chimeras.

As used herein, the term “oligonucleotide” is used interchangeable with “primer” or “polynucleotide.” The term “primer” refers to an oligonucleotide that acts as a point of initiation of DNA synthesis in a PCR reaction. A primer is usually about 15 to about 35 nucleotides in length and hybridizes to a region complementary to the target sequence.

Oligonucleotides may be synthesized and prepared by any suitable methods (such as chemical synthesis), which are known in the art. Oligonucleotides may also be conveniently available through commercial sources. One of the skilled artisans would easily optimize and identify primers flanking a polymorphic site of interest in a PCR reaction. Commercially available primers may be used to amplify a particular gene of interest for a particular SNP. A number of computer programs (e.g., Primer-Express) are readily available to design optimal primer sets. It will be apparent to one of skill in the art that the primers and probes based on the nucleic acid information provided (or publicly available with accession numbers) can be prepared accordingly.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability. “Substantially complimentary” refers to two nucleic acid strands that are sufficiently complimentary in sequence to anneal and form a stable duplex.

A person of skill in the art will know how to design PCR primers flanking the polymorphic site of interest. Synthesized oligonucleotides are typically between 20 and 26 base pairs in length with a melting point (T_{M}) of around 55 degrees. Flanking sequences for primer design can be found in the allocation files created by the International HapMap Project. These files contain a wealth of information about each SNP including observed alleles and 1,000 bp of NCBI-masked sequence for each flank. Nucleic acid template preparation

In some embodiments, the sample comprises a purified nucleic acid template (e.g., mRNA, rRNA, and mixtures thereof). Procedures for the extraction and purification of RNA from samples are well known in the art. For example, RNA can be isolated from cells using the TRIzol™ reagent (Invitrogen) extraction method. RNA quantity and quality is then determined using, for example, a Nanodrop™ spectrophotometer and an Agilent 2100 bioanalyzer.

In other embodiments, the sample is a cell lysate that is produced by lysing cells using a lysis buffer having a pH of about 6 to about 9, a zwitterionic detergent at a concentration of about 0.125% to about 2%, an azide at a concentration of about 0.3 to about 2.5 mg/ml and a protease such as proteinase K (about 1 mg/ml). After incubation at 55° C. for 15 minutes, the proteinase K is inactivated at 95° C. for 10 minutes to produce a “substantially protein free” lysate that is compatible with high efficiency PCR or reverse transcription PCR analysis.

In one embodiment, the 1× lysis reagent contains 12.5 mM Tris acetate or Tris-HCl or HEPES (4-(2-hydroxyethyl)-1-piperazineethanesulfonic acid) (pH=7-8), 0.25% (w/v) CHAPS, 0.3125 mg/ml sodium azide and proteinase K at 1 mg/ml.

The term “lysate” as used herein, refers to a liquid phase with lysed cell debris and nucleic acids.

As used herein, the term “substantially protein free” refers to a lysate where most proteins are inactivated by proteolytic cleavage by a protease. Protease may include proteinase K. Addition of proteinase K during cell lysis rapidly inactivates nucleases that might otherwise degrade the target nucleic acids. The “substantially protein free” lysate may be or may not be subjected to a treatment to remove inactivated proteins.

As used herein, the term “cells” can refer to prokaryotic or eukaryotic cells.

In one embodiment, the term “cells” can refer to microorganisms such as bacteria including, but not limited to gram positive bacteria, gram negative bacteria, acid-fast bacteria and the like. In certain embodiments, the “cells” to be tested may be collected using swab sampling of surfaces. In other embodiments, the “cells” can refer to pathogenic organisms.

In other embodiments, the sample comprises a viral nucleic acid, for example, a retroviral nucleic acid. In certain embodiments, a sample may contain a lentiviral nucleic acid such as HIV-1 or HIV-2.

As used herein, “zwitterionic detergent” refers to detergents exhibiting zwitterionic character (e.g., does not possess a net charge, lacks conductivity and electrophoretic mobility, does not bind ion-exchange resins, breaks protein-protein interactions), including, but not limited to, CHAPS, CHAPSO and Pine derivatives, e.g. preferably sulfolβines sold under the brand names Zwittergent® (Calbiochem, San Diego, Calif.) and Anzergent® (Anatrace, Inc. Maumee, Ohio).

In one embodiment, the zwitterionic detergent is CHAPS (CAS Number: 75621-03-3; available from SIGMA-ALDRICH product no. C3023-1G), an abbreviation for 3-[(3-cholamidopropyl)dimethylammonio]-1-propanesulfonate (described in further detail in U.S. Pat. No. 4,372,888) having the structure:

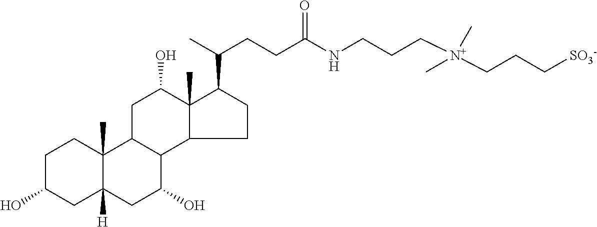

In a further embodiment, CHAPS is present at a concentration of about 0.125% to about 2% weight/volume (w/v) of the total composition. In a further embodiment, CHAPS is present at a concentration of about 0.25% to about 1% w/v of the total composition. In yet another embodiment, CHAPS is present at a concentration of about 0.4% to about 0.7% w/v of the total composition.

In other embodiments, the lysis buffer may include other non-ionic detergents such as Nonidet, Tween or Triton X-100.

As used herein, the term “lysis buffer” refers to a composition that can effectively maintain the pH value between 6 and 9, with a pKₐ at 25° C. of about 6 to about 9. The buffer described herein is generally a physiologically compatible buffer that is compatible with the function of enzyme activities and enables biological macromolecules to retain their normal physiological and biochemical functions.

Examples of buffers added to a lysis buffer include, but are not limited to, HEPES ((4-(2-hydroxyethyl)-1-piperazineethanesulfonic acid), MOPS (3-(N-morpholino)-propanesulfonic acid), N-tris(hydroxymethyl)methylglycine acid (Tricine), tris(hydroxymethyl)methylamine acid (Tris), piperazine-N,N′-bis(2-ethanesulfonic acid) (PIPES) and acetate or phosphate containing buffers (K₂HPO₄, KH₂PO₄, Na₂HPO₄, NaH₂PO₄) and the like.

The term “azide” as used herein is represented by the formula —N₃. In one embodiment, the azide is sodium azide NaN₃ (CAS number 26628-22-8; available from SIGMA-ALDRICH Product number: S2002-25G) that acts as a general bacterioside.

The term “protease,” as used herein, is an enzyme that hydrolyses peptide bonds (has protease activity). Proteases are also called, e.g., peptidases, proteinases, peptide hydrolases, or proteolytic enzymes. The proteases for use according to the invention can be of the endo-type that act internally in polypeptide chains (endopeptidases). In one embodiment, the protease can be the serine protease, proteinase K (EC 3.4.21.64; available from Roche Applied Sciences, recombinant proteinase K 50 U/ml (from Pichia pastoris) Cat. No. 03 115 887 001).

Proteinase K is used to digest protein and remove contamination from preparations of nucleic acid. Addition of proteinase K to nucleic acid preparations rapidly inactivates nucleases that might otherwise degrade the DNA or RNA during purification. It is highly-suited to this application since the enzyme is active in the presence of chemicals that denature proteins and it can be inactivated at temperatures of about 95° C. for about 10 minutes.

In one embodiment, lysis of gram positive and gram negative bacteria, such as Listeria, Salmonella, and E. Coli also requires the lysis reagent include proteinase K (1 mg/ml). Protein in the cell lysate is digested by proteinase K for 15 minutes at 55° C. followed by inactivation of the proteinase K at 95° C. for 10 minutes. After cooling, the substantially protein free lysate is compatible with high efficiency PCR amplification.

In addition to or in lieu of proteinase K, the lysis reagent can comprise a serine protease such as trypsin, chymotrypsin, elastase, subtilisin, streptogrisin, thermitase, aqualysin, plasmin, cucumisin, or carboxypeptidase A, D, C, or Y. In addition to a serine protease, the lysis solution can comprise a cysteine protease such as papain, calpain, or clostripain; an acid protease such as pepsin, chymosin, or cathepsin; or a metalloprotease such as pronase, thermolysin, collagenase, dispase, an aminopeptidase or carboxypeptidase A, B, E/H, M, T, or U. Proteinase K is stable over a wide pH range (pH 4.0-10.0) and is stable in buffers with zwitterionic detergents.

### PCR Amplification of Target Nucleic Acid Sequences

Once the primers are prepared, nucleic acid amplification can be accomplished by a variety of methods, including, but not limited to, the polymerase chain reaction (PCR), nucleic acid sequence based amplification (NASBA), ligase chain reaction (LCR), and rolling circle amplification (RCA). The polymerase chain reaction (PCR) is the method most commonly used to amplify specific target DNA sequences.

“Polymerase chain reaction,” or “PCR,” generally refers to a method for amplification of a desired nucleotide sequence in vitro. Generally, the PCR process consists of introducing a molar excess of two or more extendable oligonucleotide primers to a reaction mixture comprising a sample having the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

The term “sample” refers to any substance containing nucleic acid material.

As used herein, the term “PCR fragment” or “reverse transcriptase-PCR fragment” or “amplicon” refers to a polynucleotide molecule (or collectively the plurality of molecules) produced following the amplification of a particular target nucleic acid. A PCR fragment is typically, but not exclusively, a DNA PCR fragment. A PCR fragment can be single-stranded or double-stranded, or in a mixture thereof in any concentration ratio. A PCR fragment or RT-PCT can be about 100 to about 500 nt or more in length.

A “buffer” is a compound added to an amplification reaction which modifies the stability, activity, and/or longevity of one or more components of the amplification reaction by regulating the pH of the amplification reaction. The buffering agents of the invention are compatible with PCR amplification and site-specific RNase H cleavage activity. Certain buffering agents are well known in the art and include, but are not limited to, Tris, Tricine, MOPS (3-(N-morpholino) propanesulfonic acid), and HEPES (4-(2-hydroxyethyl)-1-piperazineethanesulfonic acid). In addition, PCR buffers may generally contain up to about 70 mM KCl and about 1.5 mM or higher MgCl₂, to about 50-200 μM each of nucleotides dATP, dCTP, dGTP and dTTP. The buffers of the invention may contain additives to optimize efficient reverse transcriptase-PCR or PCR reaction.

The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR2 or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226; and U.S. Pat. Nos. 6,268,490 and 6,794,499).

An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, Pine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll™, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. According to the invention, additives may be added to improve selectivity of primer annealing provided the additives do not interfere with the activity of RNase H.

As used herein, the term “thermostable,” as applied to an enzyme, refers to an enzyme that retains its biological activity at elevated temperatures (e.g., at 55° C. or higher), or retains its biological activity following repeated cycles of heating and cooling. Thermostable polynucleotide polymerases find particular use in PCR amplification reactions.

As used herein, an “amplifying polymerase activity” refers to an enzymatic activity that catalyzes the polymerization of deoxyribonucleotides. Generally, the enzyme will initiate synthesis at the 3′-end of the primer annealed to a nucleic acid template sequence, and will proceed toward the 5′ end of the template strand. In certain embodiments, an “amplifying polymerase activity” is a thermostable DNA polymerase.

As used herein, a thermostable polymerase is an enzyme that is relatively stable to heat and eliminates the need to add enzyme prior to each PCR cycle.

Non-limiting examples of thermostable DNA polymerases may include, but are not limited to, polymerases isolated from the thermophilic bacteria Thermus aquaticus (Taq polymerase), Thermus thermophilus (Tth polymerase), Thermococcus litoralis (Tli or VENT™ polymerase), Pyrococcus furiosus (Pfu or DEEPVENT™ polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME™ polymerase) i (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq polymerase. Many variants of Taq polymerase with enhanced properties are known and include, but are not limited to, AmpliTaq™, AmpliTaq™, Stoffel fragment, SuperTaq™, SuperTaq™ plus, LA Taq™, LApro Taq™, and EX Taq™. In another embodiment, the thermostable polymerase used in the multiplex amplification reaction of the invention is the AmpliTaq Stoffel fragment.

### Reverse Transcriptase-PCR Amplification of a RNA Target Nucleic Acid Sequence

One of the most widely used techniques to study gene expression exploits first-strand cDNA for mRNA sequence(s) as template for amplification by the PCR.

The term “reverse transcriptase activity” and “reverse transcription” refers to the enzymatic activity of a class of polymerases characterized as RNA-dependent DNA polymerases that can synthesize a DNA strand (i.e., complementary DNA, cDNA) utilizing an RNA strand as a template.

“Reverse transcriptase-PCR” of “RNA PCR” is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction.

Exemplary reverse transcriptases include, but are not limited to, the Moloney murine leukemia virus (M-MLV) RT as described in U.S. Pat. No. 4,943,531, a mutant form of M-MLV-RT lacking RNase H activity as described in U.S. Pat. No. 5,405,776, bovine leukemia virus (BLV) RT, Rous sarcoma virus (RSV) RT, Avian Myeloblastosis Virus (AMV) RT and reverse transcriptases disclosed in U.S. Pat. No. 7,883,871.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” RT PCR methods use a common buffer optimized for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV RT and Taq DNA Polymerase wherein the initial 65° C. RNA denaturation step may be omitted.

In certain embodiments, one or more primers may be labeled. As used herein, “label,” “detectable label,” or “marker,” or “detectable marker,” which are interchangeably used in the specification, refers to any chemical moiety attached to a nucleotide, nucleotide polymer, or nucleic acid binding factor, wherein the attachment may be covalent or non-covalent. Preferably, the label is detectable and renders the nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni²⁺, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also requires less sample, and reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by on-line detection in combination with these reverse transcriptase-PCR techniques has enabled accurate and precise quantitation of RNA copy number with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“TaqMan'”) or endonuclease assay (“CataCleave™”), discussed below.

### Real-Time PCR Using a Catacleave™ Probe

Post amplification amplicon detection is both laborious and time consuming. Real-time methods have been developed to monitor amplification during the PCR process. These methods typically employ fluorescently labeled probes that bind to the newly synthesized DNA or dyes whose fluorescence emission is increased when intercalated into double stranded DNA. Real time detection methodologies are applicable to PCR detection of SNPs in genomic DNA or genomic RNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red.) Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons (e.g., U.S. Pat. No. 5,925,517), TaqMan™ probes (e.g., U.S. Pat. Nos. 5,210,015 and 5,487,972), and CataCleave™ probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. TaqMan™ and CataCleave™ technologies differ from the molecular beacon in that the FRET probes employed are cleaved such that the donor and acceptor chromophores become sufficiently separated to reverse FRET.

TaqMan™ technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan™ probe binds to one strand of the amplicon at the same time that the primer binds. As the DNA polymerase extends the primer the polymerase will eventually encounter the bound TaqMan™ probe. At this time the exonuclease activity of the polymerase will sequentially degrade the TaqMan™ probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan™ works a specific amplicon can be detected only once for every cycle of PCR. Extension of the primer through the TaqMan™ target site generates a double stranded product that prevents further binding of TaqMan™ probes until the amplicon is denatured in the next PCR cycle.

U.S. Pat. No. 5,763,181, of which content is incorporated herein by reference, describes another real-time detection method (referred to as “CataCleave™”). CataCleave™ technology differs from TaqMan™ in that cleavage of the probe is accomplished by a second enzyme that does not have polymerase activity. The CataCleave™ probe has a sequence within the molecule which is a target of an endonuclease, such as, for example a restriction enzyme or RNAase. In one example, the CataCleave™ probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes an RNase H enzyme that will specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan™ probe and donor emission can be monitored. Cleavage and dissociation regenerates a site for further CataCleave™ binding. In this way it is possible for a single amplicon to serve as a target or multiple rounds of probe cleavage until the primer is extended through the CataCleave™ probe binding site.

### Labeling of a CataCleave™ Probe

The term “probe” comprises a polynucleotide that comprises a specific portion designed to hybridize in a sequence-specific manner with a complementary region of a specific nucleic acid sequence, e.g., a target nucleic acid sequence. In one embodiment, the oligonucleotide probe is in the range of 15-60 nucleotides in length. More preferably, the oligonucleotide probe is in the range of 18-30 nucleotides in length. The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices can be found in many of the references describing TaqMan™ assays or CataCleave™, described in U.S. Pat. Nos. 5,763,181, 6,787,304, and 7,112,422, of which contents are incorporated herein by reference.

In certain embodiments, the probe is “substantially complementary” to the target nucleic acid sequence.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region.

As used herein, a “selected region” refers to a polynucleotide sequence of a target DNA or cDNA that anneals with the RNA sequences of a probe. In one embodiment, a “selected region” of a target DNA or cDNA can be from 1, 2, 3, 4, 5, 6, 7, 8, 9, 10, 11, 12, 13, 14, 15, 16, 17, 18, 19, 20, 21, 22, 23, 24, 25 or more nucleotides in length.

As used herein, the site-specific RNase H cleavage refers to the cleavage of the RNA moiety of the Catacleave™ probe that is entirely complimentary to and hybridizes with a target DNA sequence to form an RNA:DNA heteroduplex.

If the RNA moiety of the Catacleave™ probe includes a single nucleotide polymorphism and the target DNA sequence includes the wild type sequence at the location of the polymorphism, formation of the RNA:DNA heteroduplex between the Catacleave™ probe and the wild-type target DNA sequence results in a single nucleotide mismatch at the location of the polymorphism that prevents cleavage of the RNA moiety of the Catacleave™ probe by RNase H.

Similarly, if the target DNA sequence includes a SNP sequence and the RNA moiety of the Catacleave™ probe includes the wild-type sequence at the location of the polymorphism, formation of the RNA:DNA heteroduplex between the Catacleave™ probe and the target DNA sequence comprising the SNP sequence results in a single nucleotide mismatch at the location of the polymorphism that prevents cleavage of the RNA moiety of the Catacleave™ probe by RNase H.

As used herein, “label” or “detectable label” of the CataCleave™ probe refers to any label comprising a fluorochrome compound that is attached to the probe by covalent or non-covalent means.

As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides energy that is absorbed by a fluorescence acceptor. The term “fluorescent acceptor” or “fluorescence acceptor” refers to either a second fluorochrome or a quenching molecule that absorbs energy emitted from the fluorescence donor. The second fluorochrome absorbs the energy that is emitted from the fluorescence donor and emits light of longer wavelength than the light emitted by the fluorescence donor. The quenching molecule absorbs energy emitted by the fluorescence donor.

Any luminescent molecule, preferably a fluorochrome and/or fluorescent quencher may be used in the practice of this invention, including, for example, Alexa Fluor™ 350, Alexa Fluor™ 430, Alexa Fluor™ 488, Alexa Fluor™ 532, Alexa Fluor™ 546, Alexa Fluor™ 568, Alexa Fluor™ 594, Alexa Fluor™ 633, Alexa Fluor™ 647, Alexa Fluor™ 660, Alexa Fluor™ 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 6501665, BODIPY TMR-X, BODIPY TR-X, Dialkylaminocoumarin, Cy5.5, Cy5, Cy3.5, Cy3, DTPA(Eu³⁺)-AMCA and TTHA(Eu³⁺)AMCA.

In one embodiment, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ position of the probe.

In one embodiment, reporter molecules are fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or 13 position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange, N-(p-(2-benzoxazolyl)phenyl)maleimide, benzoxadiazoles, stilbenes, pyrenes, and the like.

In one embodiment, reporter and quencher molecules are selected from fluorescein and rhodamine dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

### Attachment of a Catacleave™ Probe to a Solid Support

In one embodiment, the oligonucleotide probe can be attached to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include controlled pore glass, glass plates, polystyrene, avidin coated polystyrene beads cellulose, nylon, acrylamide gel and activated dextran, controlled pore glass (CPG), glass plates and high cross-linked polystyrene. These solid supports are preferred for hybridization and diagnostic studies because of their chemical stability, ease of functionalization and well defined surface area. Solid supports such as controlled pore glass (500 Å, 1000 Å) and non-swelling high cross-linked polystyrene (1000 Å) are particularly preferred in view of their compatibility with oligonucleotide synthesis.

The oligonucleotide probe may be attached to the solid support in a variety of manners. For example, the probe may be attached to the solid support by attachment of the 3′ or 5′ terminal nucleotide of the probe to the solid support. However, the probe may be attached to the solid support by a linker which serves to distance the probe from the solid support. The linker is most preferably at least 30 atoms in length, more preferably at least 50 atoms in length.

Hybridization of a probe immobilized to a solid support generally requires that the probe be separated from the solid support by at least 30 atoms, more-preferably at least 50 atoms. In order to achieve this separation, the linker generally includes a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the CataCleave™ probe is immobilized on a solid support. The CataCleave™ probe comprises a detectable label and DNA and RNA nucleic acid sequences, wherein the probe's RNA nucleic acid sequences are entirely complementary to a selected region of the target DNA sequence comprising the polymorphism and the probe's DNA nucleic acid sequences are substantially complementary to DNA sequences adjacent to the selected region of the target DNA sequence. The probe is then contacted with a sample of nucleic acids in the presence of RNase H and under conditions where the RNA sequences within the probe can form a RNA:DNA heteroduplex with the complementary DNA sequences in the PCR fragment comprising the polymorphism. RNase H cleavage of the RNA sequences within the RNA:DNA heteroduplex results in a real-time increase in the emission of a signal from the label on the probe, wherein the increase in signal indicates the presence of the polymorphism in the target DNA.

According to another embodiment of the method, the CataCleave™ probe, immobilized on a solid support, comprises a detectable label and DNA and RNA nucleic acid sequences, wherein the probe's RNA nucleic acid sequences are entirely complementary to a selected region of the target DNA sequence comprising a wild type DNA sequence at the location of the polymorphism and the probe's DNA nucleic acid sequences are substantially complementary to DNA sequences adjacent to the selected region of the target DNA sequence. The probe is then contacted with a sample of nucleic acids in the presence of RNase H and under conditions where the RNA sequences within the probe can form a RNA:DNA heteroduplex with the complementary DNA sequences in the PCR fragment comprising the polymorphism. If the target DNA sequence comprises a polymorphism, the mismatch at the location of the polymorphism in the RNA:DNA duplex prevents RNase H cleavage of the RNA sequences within the RNA:DNA heteroduplex which results in a real-time decrease in the emission of a signal from the label on the probe, wherein the decrease in signal indicates the presence of the polymorphism in the target DNA.

Immobilization of the probe to the solid support enables the target sequence hybridized to the probe to be readily isolated from the sample. In later steps, the isolated target sequence may be separated from the solid support and processed (e.g., purified, amplified) according to methods well known in the art depending on the particular needs of the researcher.

### RNase H Cleavage of the Catacleave™ Probe

RNase H hydrolyzes RNA in RNA-DNA hybrids. First identified in calf thymus, RNase H has subsequently been described in a variety of organisms. Indeed, RNase H activity appears to be ubiquitous in eukaryotes and bacteria. Although RNase Hs form a family of proteins of varying molecular weight and nucleolytic activity, substrate requirements appear to be similar for the various isotypes. For example, most RNase Hs studied to date function as endonucleases and require divalent cations (e.g., Mg²⁺, Mn²⁺) to produce cleavage products with 5′ phosphate and 3′ hydroxyl termini.

In prokaryotes, RNase H have been cloned and extensively characterized (see Crooke, et al., (1995) Biochem J, 312 (Pt 2), 599-608; Lima, et al., (1997) J Biol Chem, 272, 27513-27516; Lima, et al., (1997) Biochemistry, 36, 390-398; Lima, et al., (1997) J Biol Chem, 272, 18191-18199; Lima, et al., (2007) Mol Pharmacol, 71, 83-91; Lima, et al., (2007) Mol Pharmacol, 71, 73-82; Lima, et al., (2003) J Biol Chem, 278, 14906-14912; Lima, et al., (2003) J Biol Chem, 278, 49860-49867; Itaya, M., Proc. Natl. Acad. Sci. USA, 1990, 87, 8587-8591). For example, E. coli RNase HII is 213 amino acids in length whereas RNase HI is 155 amino acids long. E. coli RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HI enzymes are reported to have molecular weights in the 68-90 kDa range, be activated by either Mn²⁺ or Mg²⁺ and be insensitive to sulfhydryl agents. In contrast, RNase HII enzymes have been reported to have molecular weights ranging from 31-45 kDa, to require Mg²⁺ to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108)

An enzyme with RNase HII characteristics has also been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

A detailed comparison of RNases from different species is reported in Ohtani N, Haruki M, Morikawa M, Kanaya S. J Biosci Bioeng. 1999; 88(1):12-9.

Examples of RNase H enzymes, which may be employed in the embodiments, also include, but are not limited to, thermostable RNase H enzymes isolated from thermophilic organisms such as Pyrococcus furiosus, Pyrococcus horikoshi, Thermococcus litoralis or Thermus thermophilus.

Other RNase H enzymes that may be employed in the embodiments are described in, for example, U.S. Pat. No. 7,422,888 to Uemori or the published U.S. Patent Application No. 2009/0325169 to Walder, the contents of which are incorporated herein by reference.

In one embodiment, an RNase H enzyme is a thermostable RNase H with 40%, 50%, 60%, 70%, 80%, 90%, 95% or 99% homology with the amino acid sequence of Pfu RNase HII (SEQ ID NO: 13), shown below.

| (SEQ ID NO: 13) | |
|---|---|
| MKIGGIDEAG RGPAIGPLVV ATVVVDEKNI EKLRNIGVKD SKQLTPHERK NLFSQITSIA | 60 |
| DDYKIVIVSP EEIDNRSGTM NELEVEKFAL ALNSLQIKPA LIYADAADVD ANRFASLIER | 120 |
| RLNYKAKIIA EHKADAKYPV VSAASILAKV VRDEEIEKLK KQYGDFGSGY PSDPKTKKWL | 180 |
| EEYYKKHNSF PPIVRRTWET VRKIEESIKA KKSQLTLDKF FKKP | |

The homology can be determined using, for example, a computer program DNASIS-Mac (Takara Shuzo), a computer algorithm FASTA (version 3.0; Pearson, W. R. et al., Pro. Natl. Acad. Sci., 85:2444-2448, 1988) or a computer algorithm BLAST (version 2.0, Altschul et al., Nucleic Acids Res. 25:3389-3402, 1997)

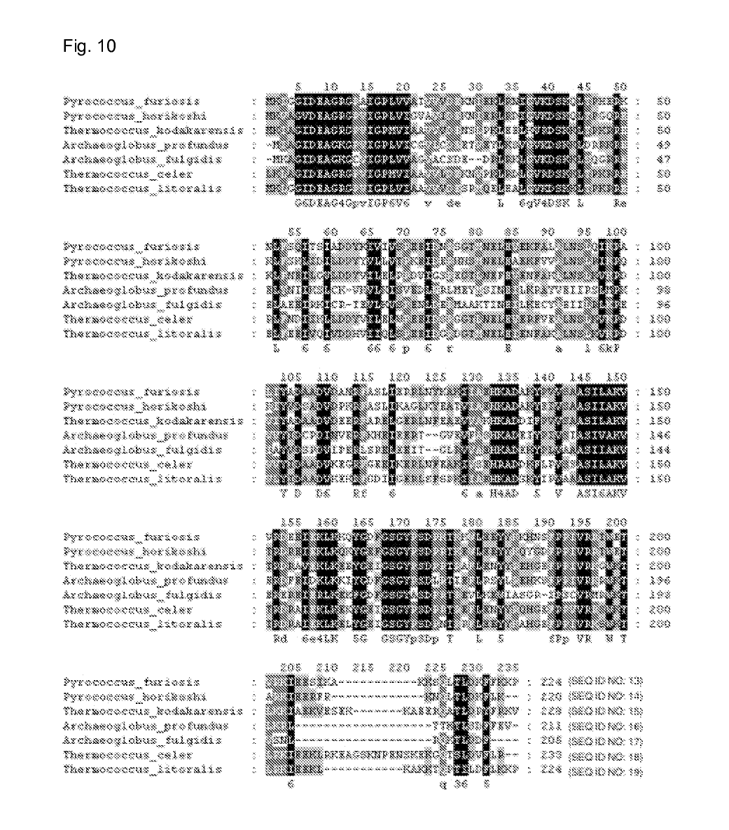

In another embodiment, an RNase H enzyme is a thermostable RNase H with at least one or more homology regions 1-4 corresponding to positions 5-20, 33-44, 132-150, and 158-173 of SEQ ID NO: 13. These homology regions were defined by sequence alignment of Pyrococcus furiosis, Pyrococcus horikoshi, Thermococcus kodakarensis, Archeoglobus profundus, Archeoglobus fulgidis, Thermococcus celer and Thermococcus litoralis RNase HII polypeptide sequences (see FIG. 10).

HOMOLOGY REGION 1:
(SEQ ID NO: 20; corresponding to
positions 5-20 of SEQ ID NO: 13)
GIDEAG RGPAIGPLVV
HOMOLOGY REGION 2:
(SEQ ID NO: 21; corresponding to
positions 33-44 of SEQ ID NO: 13)
LRNIGVKD SKQL
HOMOLOGY REGION 3:
(SEQ ID NO: 22; corresponding to
positions 132-150 of SEQ ID NO: 13)
HKADAKYPV VSAASILAKV
HOMOLOGY REGION 4:
(SEQ ID NO: 23; corresponding to
positions 158-173 of SEQ ID NO: 13)
KLK KQYGDFGSGY PSD

In one embodiment, an RNase H enzyme is a thermostable RNase H with at least one of the homology regions having 50%, 60%. 70%, 80%, 90% sequence identity with a polypeptide sequence of SEQ ID NOs: 20, 21, 22 or 23.

In another embodiment, an RNase H enzyme is a thermostable RNase H with 40%, 50%, 60%, 70%, 80%, 90%, 95% or 99% homology with the amino acid sequence of Thermus thermophilus RNase HI (SEQ ID NO: 25), shown below.

(SEQ ID NO: 25)
MNPSPRKRVA LFTDGACLGN PGPGGWAALL RFHAHEKLLS GGEACTTNNR MELKAAIEGL
KALKEPCEVD LYTDSHYLKK AFTEGWLEGW RKRGWRTAEG KPVKNRDLWE ALLLAMAPHR
VRFHFVKGHT GHPENERVDR EARRQAQSQA KTPCPPRAPT LFHEEA

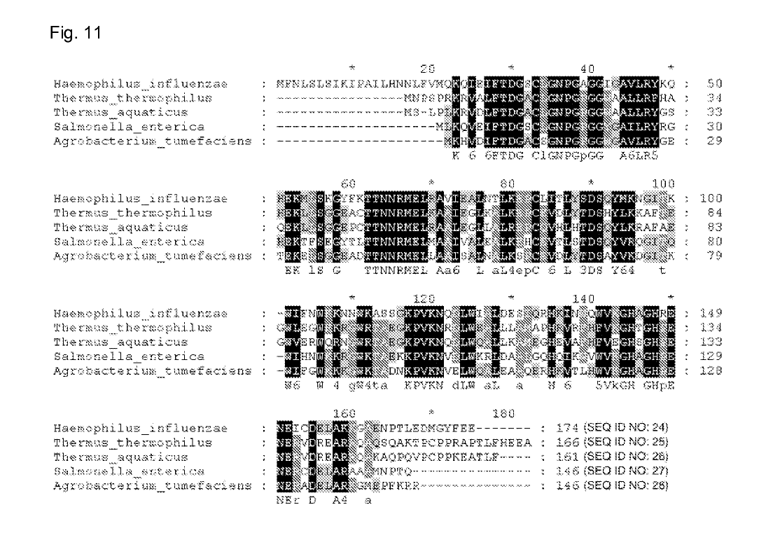

In another embodiment, an RNase H enzyme is a thermostable RNase H with at least one or more homology regions 5-8 corresponding to positions 23-48, 62-69, 117-121 and 141-152 of SEQ ID NO: 25. These homology regions were defined by sequence alignment of Haemophilus influenzae, Thermus thermophilis, Thermus acquaticus, Salmonella enterica and Agrobacterium tumefaciens RNase HI polypeptide sequences (see FIG. 11).

HOMOLOGY REGION 5:
(SEQ ID NO: 29; corresponding to
positions 23-48 of SEQ ID NO: 25)
K*V*LFTDG*C*GNPG*GG*ALLRY
HOMOLOGY REGION 6:
(SEQ ID NO: 30; corresponding to
positions 62-69 of SEQ ID NO: 25)
TTNNRMEL
HOMOLOGY REGION 7:
(SEQ ID NO: 31; corresponding to
positions 117-121 of SEQ ID NO: 25)
KPVKN
HOMOLOGY REGION 8:
(SEQ ID NO: 32; corresponding to
positions 141-152 of SEQ ID NO: 25)
FVKGH*GH*ENE

In another embodiment, an RNase H enzyme is a thermostable RNase H with at least one of the homology regions 4-8 having 50%, 60%. 70%, 80%, 90% sequence identity with a polypeptide sequence of SEQ ID NOs: 29, 30, 31 or 32.

The terms “sequence identity” as used herein refers to the extent that sequences are identical or functionally or structurally similar on a amino acid to amino acid basis over a window of comparison. Thus, a “percentage of sequence identity”, for example, can be calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity.

In certain embodiments, the RNase H can be modified to produce a hot start “inducible” RNase H.

The term “modified RNase H,” as used herein, can be an RNase H reversely coupled to or reversely bound to an inhibiting factor that causes the loss of the endonuclease activity of the RNase H. Release or decoupling of the inhibiting factor from the RNase H restores at least partial or full activity of the endonuclease activity of the RNase H. About 30-100% of its activity of an intact RNase H may be sufficient. The inhibiting factor may be a ligand or a chemical modification. The ligand can be an antibody, an aptamer, a receptor, a cofactor, or a chelating agent. The ligand can bind to the active site of the RNase H enzyme thereby inhibiting enzymatic activity or it can bind to a site remote from the RNase's active site. In some embodiment, the ligand may induce a conformational change. The chemical modification can be a crosslinking (for example, by formaldehyde) or acylation. The release or decoupling of the inhibiting factor from the RNase H may be accomplished by heating a sample or a mixture containing the coupled RNase H (inactive) to a temperature of about 65° C. to about 95° C. or higher, and/or lowering the pH of the mixture or sample to about 7.0 or lower.

As used herein, a hot start “inducible” RNase H activity refers to the herein described modified RNase H that has an endonuclease catalytic activity that can be regulated by association with a ligand. Under permissive conditions, the RNase H endonuclease catalytic activity is activated whereas at non-permissive conditions, this catalytic activity is inhibited. In some embodiments, the catalytic activity of a modified RNase H can be inhibited at temperature conducive for reverse transcription, i.e. about 42° C., and activated at more elevated temperatures found in PCR reactions, i.e. about 65° C. to 95° C. A modified RNase H with these characteristics is said to be “heat inducible.”

In other embodiments, the catalytic activity of a modified RNase H can be regulated by changing the pH of a solution containing the enzyme.

As used herein, a “hot start” enzyme composition refers to compositions having an enzymatic activity that is inhibited at non-permissive temperatures, i.e. from about 25° C. to about 45° C. and activated at temperatures compatible with a PCR reaction, e.g. about 55° C. to about 95° C. In certain embodiment, a “hot start” enzyme composition may have a ‘hot start’ RNase H and/or a ‘hot start’ thermostable DNA polymerase that are known in the art.

Crosslinking of RNase H enzymes can be performed using, for example, formaldehyde. In one embodiment, a thermostable RNase H is subjected to controlled and limited crosslinking using formaldehyde. By heating an amplification reaction composition, which comprises the modified RNase H in an active state, to a temperature of about 95° C. or higher for an extended time, for example about 15 minutes, the crosslinking is reversed and the RNase H activity is restored.

In general, the lower the degree of crosslinking, the higher the endonuclease activity of the enzyme is after reversal of crosslinking The degree of crosslinking may be controlled by varying the concentration of formaldehyde and the duration of crosslinking reaction. For example, about 0.2% (w/v), about 0.4% (w/v), about 0.6% (w/v), or about 0.8% (w/v) of formaldehyde may be used to crosslink an RNase H enzyme. About 10 minutes of crosslinking reaction using 0.6% formaldehyde may be sufficient to inactivate RNase HII from Pyrococcus furiosus.

The crosslinked RNase H does not show any measurable endonuclease activity at about 37° C. In some cases, a measurable partial reactivation of the crosslinked RNase H may occur at a temperature of around 50° C., which is lower than the PCR denaturation temperature. To avoid such unintended reactivation of the enzyme, it may be required to store or keep the modified RNase H at a temperature lower than 50° C. until its reactivation.

In general, PCR requires heating the amplification composition at each cycle to about 95° C. to denature the double stranded target sequence which will also release the inactivating factor from the RNase H, partially or fully restoring the activity of the enzyme.

RNase H may also be modified by subjecting the enzyme to acylation of lysine residues using an acylating agent, for example, a dicarboxylic acid. Acylation of RNase H may be performed by adding cis-aconitic anhydride to a solution of RNase H in an acylation buffer and incubating the resulting mixture at about 1-20° C. for 5-30 hours. In one embodiment, the acylation may be conducted at around 3-8° C. for 18-24 hours. The type of the acylation buffer is not particularly limited. In an embodiment, the acylation buffer has a pH of between about 7.5 to about 9.0.

The activity of acylated RNase H can be restored by lowering the pH of the amplification composition to about 7.0 or less. For example, when Tris buffer is used as a buffering agent, the composition may be heated to about 95° C., resulting in the lowering of pH from about 8.7 (at 25° C.) to about 6.5 (at 95° C.).

The duration of the heating step in the amplification reaction composition may vary depending on the modified RNase H, the buffer used in the PCR, and the like. However, in general, heating the amplification composition to 95° C. for about 30 seconds—4 minutes is sufficient to restore RNase H activity. In one embodiment, using a commercially available buffer and one or more non-ionic detergents, full activity of Pyrococcus furiosus RNase HII is restored after about 2 minutes of heating.

RNase H activity may be determined using methods that are well in the art. For example, according to a first method, the unit activity is defined in terms of the acid-solubilization of a certain number of moles of radiolabeled polyadenylic acid in the presence of equimolar polythymidylic acid under defined assay conditions (see Epicentre Hybridase thermostable RNase HI). In the second method, unit activity is defined in terms of a specific increase in the relative fluorescence intensity of a reaction containing equimolar amounts of the probe and a complementary template DNA under defined assay conditions.

### Real-Time Detection of SNPs

The labeled oligonucleotide probe may be used as a probe for the real-time detection of SNPs in a target nucleic acid.

A CataCleave™ oligonucleotide probe is first synthesized with DNA and RNA sequences that are complimentary to sequences found within a PCR amplicon that encompasses a single nucleotide polymorphism (SNP). The probe can be labeled, for example, with a FRET pair, for example, a fluorescein molecule at one end of the probe and a rhodamine quencher molecule at the other end. The probe can be synthesized to be substantially complementary to a target nucleic acid sequence encompassing the location of the selected SNP.

In certain embodiments, the RNA sequence of the probe can be engineered to have a sequence that is complimentary to the wild type sequence.

In other embodiments, the RNA sequence of the probe is be engineered to have a sequence that is complimentary to the SNP sequence.

In one embodiment, real-time nucleic acid amplification is performed on a target polynucleotide in the presence of a thermostable nucleic acid polymerase, a RNase H activity, a pair of PCR amplification primers capable of hybridizing to the target polynucleotide encompassing the SNP, and a labeled CataCleave™ oligonucleotide probe. During the real-time PCR reaction, RNase H cleavage of the RNA:DNA heteroduplex probe formed between the RNA moiety of the CataCleave™ oligonucleotide probe and the SNP present in the PCR amplicon leads to the separation of the fluorescent donor from the fluorescent quencher and results in the real-time increase in fluorescence of the probe corresponding to the real-time detection of the SNP in the PCR amplicon and hence the target DNA.

In certain embodiments, the RNA moiety of the probe comprises the wild-type sequence at the location of the SNP in the target DNA sequence. Hence, upon hybridization of the probe with the PCR amplicon encompassing the SNP, a RNA:DNA heteroduplex forms having a single nucleotide mismatch at the location of the SNP that cannot be cleaved by an RNase H activity.

In other embodiments, the RNA moiety of the probe comprises the complementary SNP sequence at the location of the SNP in the target DNA sequence. Hence, upon hybridization of the probe with the PCR amplicon encompassing the SNP, a RNA:DNA heteroduplex forms without a mismatch at the location of the SNP that can be cleaved by an RNase H activity.

### Kits

The disclosure herein also provides for a kit format which comprises a package unit having one or more reagents for the real-time detection of SNP in a target nucleic acid. The kit may also contain one or more of the following items: buffers, instructions, and positive or negative controls. Kits may include containers of reagents mixed together in suitable proportions for performing the methods described herein. Reagent containers preferably contain reagents in unit quantities that obviate measuring steps when performing the subject methods.

Kits may also contain reagents for real-time PCR including, but not limited to, a thermostable polymerase, RNase H, primers selected to amplify a region encompassing the location of a SNP and a labeled CataCleave™ oligonucleotide probe that anneals to the real-time PCR product and allow for the detection of the SNP according to the methodology described herein. Kits may comprise reagents for the detection of SNPs within a single gene or locus or SNPs amongst two more genes or loci. In another embodiment, the kit reagents further comprised reagents for the extraction of genomic DNA or RNA from a biological sample. Kit reagents may also include reagents for reverse transcriptase-PCR analysis where applicable.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

### EXAMPLES

The following examples set forth methods for using the RNase H enzyme composition according to the present invention. It is understood that the steps of the methods described in these examples are not intended to be limiting. Further objectives and advantages of the present invention other than those set forth above will become apparent from the examples which are not intended to limit the scope of the present invention.

### Example 1

### Isothermal Detection of a Synthetic SNP in the Salmonella invA Gene

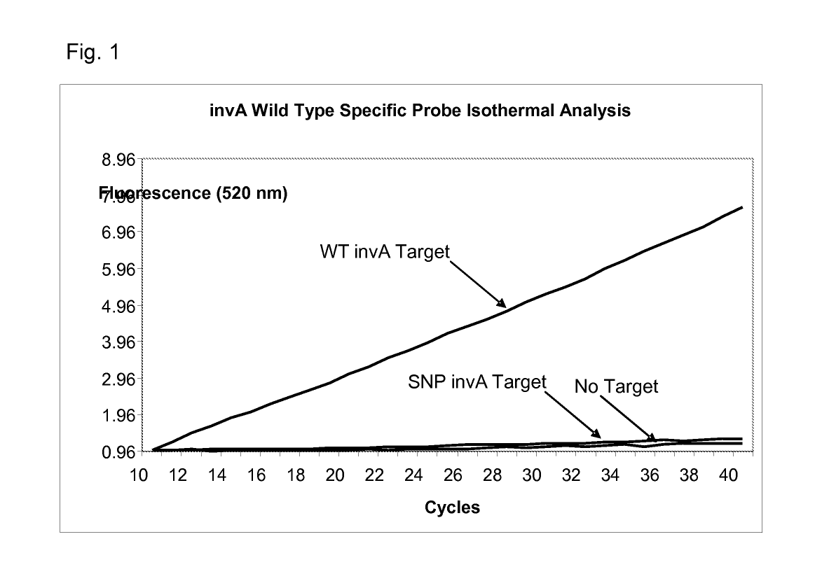

An artificial single nucleotide polymorphism (SNP) was created in the invA gene (SEQ ID NO: 33) of Salmonella to test the ability of CataCleave™ probes to differentiate single nucleotide sequence differences within a target DNA sequence. The single nucleotide change created a T to G transversion at position 116 of the Salmonella invA coding sequence (SEQ ID NO: 33). Two similar 19 nucleotide CataCleave™ probes, each dually labeled to create FRET pairs were designed such that they would base pair across the region of invA containing the SNP nucleotide. The wild type specific probe inv-CCProbe2 (SEQ ID NO: 1) contained perfect complementarity with the wild type sequence of invA and the SNP specific probe inv-CCProbe2-2C (SEQ ID NO: 2) contained perfect complementarity with the mutant form of invA. The probes were designed such that the 2^{nd} (relative to the 5′ end of the probe) of the 4 RNA bases of the CataCleave™ probe would base pair at the position of the SNP nucleotide. Two DNA oligonucleotides were synthesized, inv2-Target1 (SEQ ID NO: 3), which was complementary to the wild type specific invA probe and inv2-Target8 (SEQ ID NO: 4), which was complementary to the SNP specific invA probe. Isothermal processing reactions were performed using RNase HI to evaluate the ability of the two probes to differentiate the single nucleotide mismatches. The final concentrations of each component in the reactions were as follows, 200 nM probe, 0.4 nM target oligonucleotide, 10 mM Tris acetate pH 8.6, 50 mM potassium acetate, 2.5 mM magnesium acetate, 1 mM DTT and 2.5 u Hybridase thermostable RNase HI (Epicentre). The reactions were incubated for 60 min at 55° C. collecting fluorescence data every minute. FIG. 1 shows the fluorescent signal generated when inv-CCProbe2 (SEQ ID NO: 1) was reacted with either inv2-Target1 (SEQ ID NO: 3) or inv2-Target8 (SEQ ID NO: 4). When inv-CCProbe2 was incubated with inv2-Target1 fluorescent signal increased linearly indicating that RNase HI recognizes and cleaves the probe perfectly paired with the oligonucleotide. When inv-CCProbe2 was incubated with inv2-Target8 very little fluorescent signal was generated indicating that the mismatched oligonucleotides were a poor target for RNase HI.

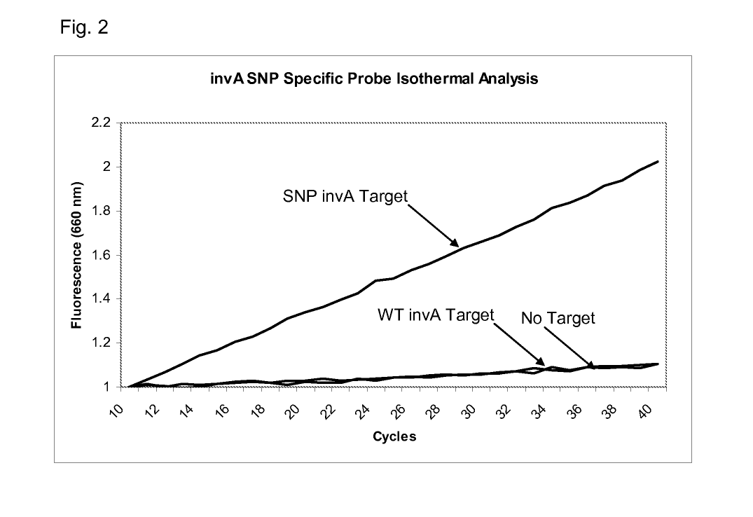

FIG. 2 shows the fluorescent signal generated when inv-CCProbe2-2C (SEQ ID NO: 2) was reacted with either inv2-Target1 (SEQ ID NO: 3) or inv2-Target8 (SEQ ID NO: 4). Inv-CCProbe2-2C cleavage was achieved by RNase HI when it was incubated with the perfectly paired inv2-Target8 indicated by the increase in fluorescence. Little fluorescent signal was achieved upon incubation with the wild type invA target indicating poor probe cleavage of the mismatched pair.

### Example 2

### Real-Time PCR Detection of a Synthetic SNP in the Salmonella invA Gene

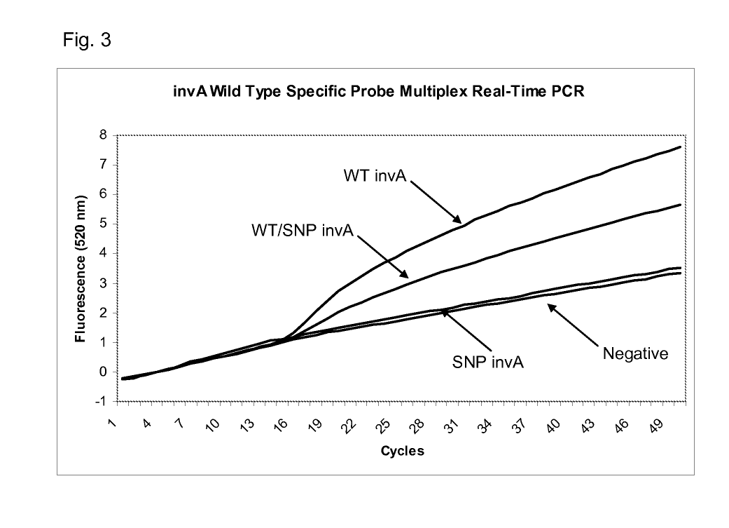

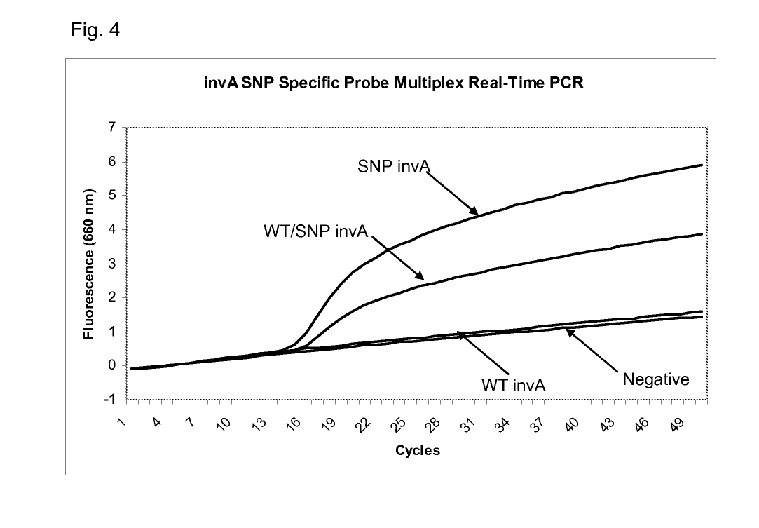

Plasmid DNAs containing 267 nucleotides of invA sequence encompassing either the wild type or mutant base (described above) were synthesized. Forty pg of the wild type plasmid, mutant plasmid or mix of the two plasmids was used as template in multiplex real-time PCR reactions containing differentially labeled probes complementary to the wild type sequence or the mutant sequence. The final concentrations of each component in the reactions were as follows, 800 nM forward primer Salmonella-F1 (SEQ ID NO: 5), 800 nM reverse primer sal-invR2 (SEQ ID NO: 6), 200 nM wild type specific probe inv-CCProbe2 (SEQ ID NO: 1), 200 nM SNP specific probe inv-CCProbe2-2C (SEQ ID NO: 2), 80 uM each dNTP, 10 mM Tris acetate pH 8.6, 50 mM potassium acetate, 2.5 mM magnesium acetate, 1 mM DTT, 2.5 u Platinum Taq DNA Polymerase (Life Technologies) and 2.5 u Hybridase thermostable RNase HI (Epicentre). The PCR reactions were incubated for 2 min at 95 C to activate the hot start DNA polymerase followed by 40 cycles of 95 C for 10 seconds and 60 C for 20 seconds. FIG. 3 shows the fluorescent signal generated during PCR from the FAM labeled inv-CCProbe2 (SEQ ID NO: 1) and FIG. 4 shows the fluorescent signal generated during PCR from the TYE665 labeled inv-CCProbe2-2C (SEQ ID NO: 2). The fluorescence curves for these PCR reactions indicate that each probe was capable of detecting amplification of the perfectly complementary target and did not detect amplification of target containing a single mismatch.

### Example 3

### Isothermal Detection of the A1 and A2 Alleles of the Bovine β Casein Gene

There are two entirely natural variants or forms of β casein protein in the milk of dairy cows, called A2 and A1β casein. The difference between A1 and A2β caseins is in a single amino acid, at position 67. In the A1 variant the base is a T and in the A2 variant the base is a G. The A1 variant β casein in cow's milk is unique amongst all mammalian β caseins, in having a histidine amino acid at this position. Other species milk contains β casein that can be considered A2 like, as they have a proline amino acid at this equivalent position in their β casein chains. Water buffalo, yak, goat as well as human breast milk all contain the A2-like form of β casein.

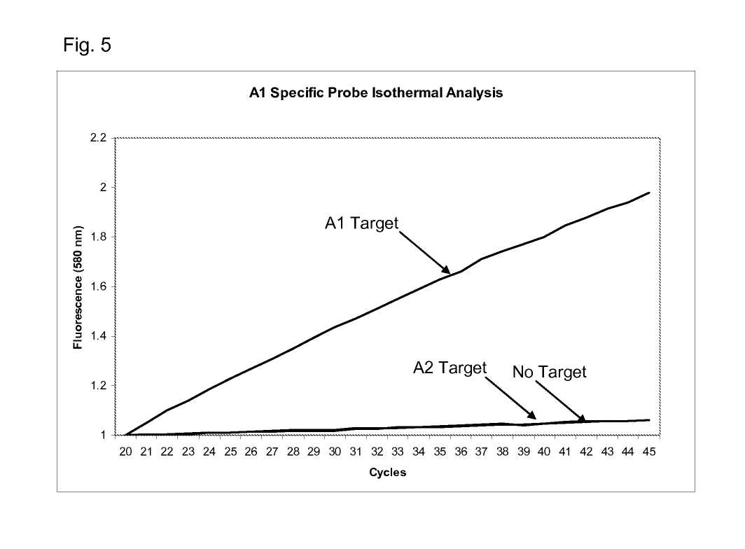

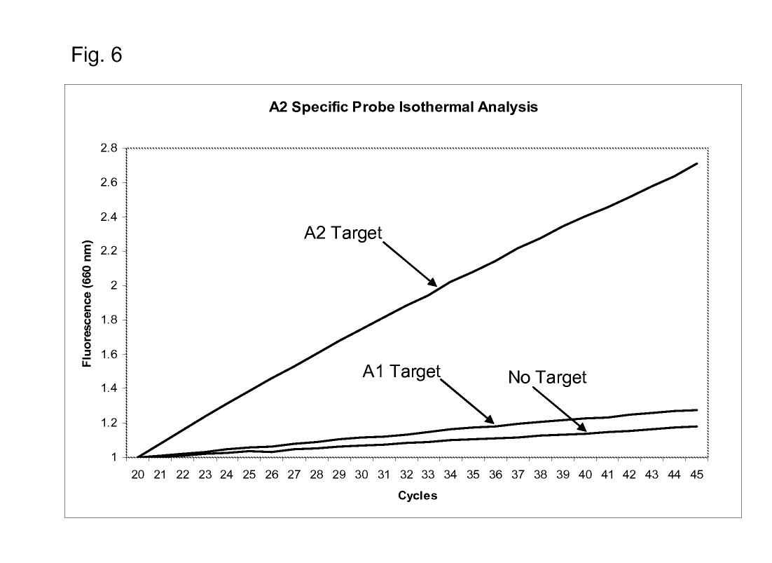

In this example two similar 19 nucleotide CataCleave™ probes, each dually labeled to create FRET pairs were designed such that they would base pair across the location of the A1/A2 SNP nucleotide in the bovine β casein gene. A1-CCProbe2-RC (SEQ ID NO:7) base pairs perfectly with the A1 allele and A2-CCProbe1-RC (SEQ ID NO: 8) base pairs perfectly with the A2 allele. A1-CCProbe2-RC was designed such that the 2^{nd} (relative to the 5′ end of the probe) of the 4 RNA bases of the CataCleave™ probe would base pair at the position of the SNP nucleotide. The A2-CCProbe1-RC was designed such that the 1^{st} (relative to the 5′ end of the probe) of the 4 RNA bases of the CataCleave™ probe would base pair at the position of the SNP nucleotide. Two DNA oligonucleotides were synthesized, A1-Target-RC (SEQ ID NO: 9) representing the A1 allele and A2-Target-RC (SEQ ID NO: 10) representing the A2 allele. Isothermal processing reactions were performed using RNase HI to evaluate the ability of the two probes to differentiate the single nucleotide difference. The final concentrations of each component in the reactions were as follows, 200 nM probe, 0.4 nM target oligonucleotide, 10 mM Tris acetate pH 8.6, 50 mM potassium acetate, 2.5 mM magnesium acetate, 1 mM DTT and 2.5 u Hybridase thermostable RNase HI (Epicentre). The reactions were incubated for 60 min at 55 C collecting fluorescence data every minute. FIG. 5 shows the fluorescent signal generated when A1-CCProbe2-RC (SEQ ID NO: 7) was reacted with either A1-Target-RC (SEQ ID NO: 9) or A2-Target-RC (SEQ ID NO: 10). When A1-CCProbe2-RC was incubated with the A1-Target-RC, fluorescent signal increased linearly indicating that RNase HI recognizes and cleaves the perfectly paired oligonucleotides. When A1-CCProbe2-RC was incubated with the A2-Target-RC very little fluorescent signal was generated indicating that the mismatched oligonucleotides are a poor target for RNase HI. FIG. 6 shows the fluorescent signal generated when A2-CCProbe1-RC (SEQ ID NO: 8) was reacted with either A1-Target-RC (SEQ ID NO: 9) or A2-Target-RC (SEQ ID NO: 10). A2-CCProbe1-RC cleavage was achieved by RNase HI when it was incubated with A2-Target-RC but no cleavage occurred upon incubation with the A1-Target-RC.

### Example 4

### Real-Time PCR Detection of the A1 and A2 Alleles of the Bovine β Casein Gene

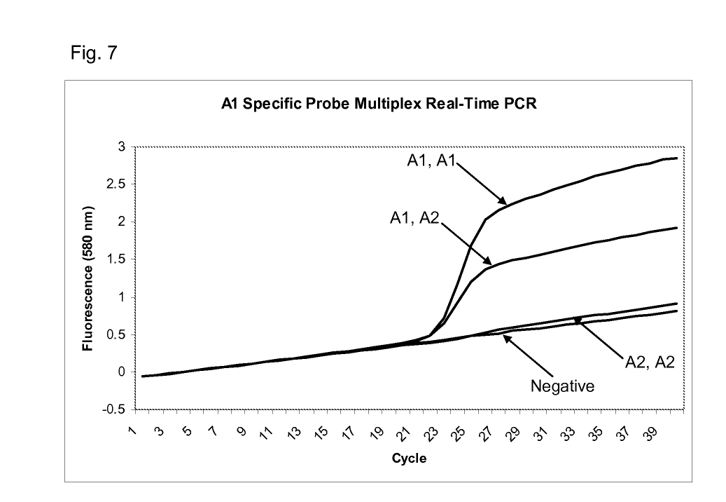

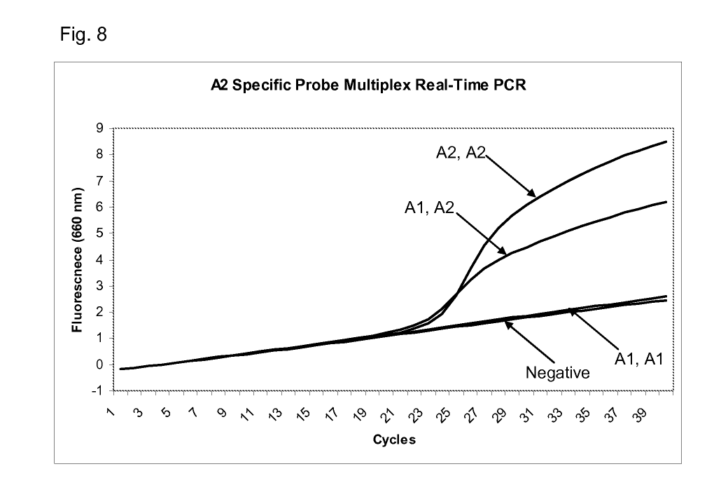

Three sets of dairy bull DNAs were used for genotyping using CataCleave™ based SNP detection. The three DNAs were previously genotyped by sequencing and were known to represent the three possible genotypes of the β casein gene, A1/A1, A2/A2 and A1/A2. The DNA was extracted from bull semen as described previously by Heyen et al. Two hundred ng of A1/A1 genotype, A2/A2 genotype or A1/A2 genotype genomic DNA was used as template in multiplex real-time PCR reactions containing both of the differentially labeled A1-CCProbe2-RC (SEQ ID NO: 7) and A2-CCProbe1-RC (SEQ ID NO: 8). The final concentrations of each component in the reactions were as follows, 800 nM forward primer A2D-F (SEQ ID NO: 11), 800 nM reverse primer A2D-R-150 (SEQ ID NO: 12), 200 nM A1-CCProbe2-RC (SEQ ID NO: 7), 200 nM A2-CCProbe1-RC (SEQ ID NO: 8), 80 uM each dNTP, 10 mM Tris acetate pH 8.6, 50 mM potassium acetate, 2.5 mM magnesium acetate, 1 mM DTT, 2.5 u Platinum Taq DNA Polymerase (Life Technologies) and 2.5 u Hybridase thermostable RNase HI (Epicentre). The PCR reactions were incubated for 2 min at 95 C to activate the hot start DNA polymerase followed by 40 cycles of 95 C for 10 seconds and 60 C for 30 seconds. FIG. 7 shows the fluorescent signal generated during PCR from the TYE563 labeled A1-CCProbe2-RC (SEQ ID NO: 7) and FIG. 8 shows the fluorescent signal generated during PCR from the TYE665 labeled A2-CCProbe1-RC (SEQ ID NO: 8). The fluorescence curves for these PCR reactions indicate that each probe was capable of detecting amplification of the perfectly complementary targets and did not detect amplification of target containing a single mismatch.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

1. A method for the real-time detection of a polymorphism in a target DNA, comprising the steps of:
a) providing a sample to be tested for the presence of a target DNA having a polymorphism;
b) providing a pair of amplification primers that can anneal to the target DNA, wherein a first amplification primer anneals upstream of the location of the polymorphism and the second amplification primer anneals downstream of the location of the polymorphism;
c) providing a probe comprising a detectable label and DNA and RNA nucleic acid sequences, wherein the probe's RNA nucleic acid sequences are entirely complementary to a selected region of the target DNA sequence comprising the polymorphism and the probe's DNA nucleic acid sequences are substantially complementary to DNA sequences adjacent to the selected region of the target DNA sequence;
d) amplifying a PCR fragment between the first and second amplification primers in the presence of an amplifying polymerase activity, amplification buffer; an RNase H activity and the probe under conditions where the RNA sequences within the probe can form a RNA:DNA heteroduplex with the complementary DNA sequences in the PCR fragment comprising the polymorphism; and
e) detecting a real-time increase in the emission of a signal from the label on the probe,
wherein the increase in signal indicates the presence of the polymorphism in the target DNA.

2. The method of claim 1, wherein the real-time increase in the emission of the signal from the label on the probe results from the RNase H cleavage of the probe's RNA sequences in the RNA:DNA heteroduplex.

3. The method of claim 1, wherein the RNA nucleic acid sequence of the probe comprises a sequence that is complimentary to the polymorphism in the target DNA.

4. The method of claim 1, wherein the polymorphism is a single nucleotide polymorphism (SNP).

5. The method of claim 1, wherein the DNA and RNA sequences of the probe are covalently linked.

6. The method of claim 1, wherein the detectable label on the probe is a fluorescent label.

7. The method of claim 6, wherein the fluorescent label comprises a FRET pair.

8. The method of claim 1, wherein the PCR fragment is linked to a solid support.

9. A method for the real-time detection of a polymorphism in a target DNA, comprising steps of:
a) providing a sample to be tested for the presence of a target DNA having a polymorphism;
b) providing a pair of amplification primers that can anneal to the target DNA, wherein a first amplification primer anneals upstream of the location of the polymorphism and the second amplification primer anneals downstream of the location of the polymorphism;
c) providing a probe comprising a detectable label and DNA and RNA nucleic acid sequences, wherein the probe's RNA nucleic acid sequences are entirely complementary to a selected region of the target DNA sequence comprising a wild type DNA sequence at the location of the polymorphism and the probe's DNA nucleic acid sequences are substantially complementary to DNA sequences adjacent to the selected region of the target DNA sequence;
d) amplifying a PCR fragment between the first and second amplification primers in the presence of an amplifying polymerase activity, amplification buffer; an RNase H activity and the probe under conditions where the RNA sequences within the probe can form a RNA:DNA heteroduplex with the complementary DNA sequences in the PCR fragment comprising the polymorphism; and
e) detecting a real-time decrease in the emission of a signal from the label on the probe,
wherein the decrease in signal indicates the presence of the polymorphism in the target DNA.

10. A method for the real-time detection of a polymorphism in a RNA target, comprising the steps of:
a) providing a sample to be tested for a RNA target having a polymorphism;
b) providing a pair of amplification primers that can anneal to a cDNA of the RNA target, wherein a first amplification primer anneals upstream of the location of the polymorphism and the second amplification primer anneals downstream of the location of the polymorphism;
c) providing a probe comprising a detectable label and DNA and RNA nucleic acid sequences, wherein the probe's RNA nucleic acid sequences are entirely complementary to a selected region of the cDNA comprising the polymorphism and the probe's DNA nucleic acid sequences are substantially complementary to DNA sequences adjacent to the selected region of the cDNA;
d) amplifying a reverse transcriptase-PCR fragment between the first and second amplification primers in the presence of a reverse transcriptase activity, an amplifying polymerase activity, a reverse transcriptase-PCR buffer; an RNase H activity and the probe and under conditions where the RNA sequences within the probe can form a RNA:DNA heteroduplex with complementary sequences in the RT-PCR DNA fragment comprising the polymorphism; and
e) detecting a real-time increase in the emission of a signal from the label on the probe,
wherein the increase in signal indicates the presence of the polymorphism in the RNA target.

11. The method of claim 10, wherein the real-time increase in the emission of the signal from the label on the probe results from the RNase H cleavage of the probe's RNA sequences in the RNA:DNA heteroduplex.

12. The method of claim 10, wherein the RNA nucleic acid sequence of the probe comprises an RNA sequence that is complementary to the cDNA at the location of the polymorphism in the target RNA.

13. The method of claim 10, wherein the polymorphism is a single nucleotide polymorphism (SNP).

14. The method of claim 10, wherein the RNA target is an mRNA transcript.

15. The method of claim 10, wherein the DNA and RNA sequences of the probe are covalently linked.

16. The method of claim 10, wherein the detectable label on the probe is a fluorescent label.

17. The method of claim 16, wherein the fluorescent label comprises a FRET pair.

18. The method of claim 10, wherein the probe or PCR fragment is linked to a solid support.

19. The method of claim 10, wherein the RNase H activity is the activity of a hot start, thermostable RNase H.

20. A method for the real-time detection of a polymorphism in a RNA target, comprising the steps of:
a) providing a sample to be tested for the RNA target having a polymorphism;
b) providing a pair of amplification primers that can anneal to a cDNA of the RNA target, wherein a first amplification primer anneals upstream of the location of the polymorphism and the second amplification primer anneals downstream of the location of the polymorphism;
c) providing a probe comprising a detectable label and DNA and RNA nucleic acid sequences, wherein the probe's RNA nucleic acid sequences are entirely complementary to a selected region of the cDNA comprising a wild type DNA sequence at the location of the polymorphism and the probe's DNA nucleic acid sequences are substantially complementary to DNA sequences adjacent to the selected region of the cDNA;
d) amplifying a reverse transcriptase-PCR fragment between the first and second amplification primers in the presence of a reverse transcriptase activity, an amplifying polymerase activity, a reverse transcriptase-PCR buffer; an RNase H activity and the probe and under conditions where the RNA sequences within the probe can form a RNA:DNA heteroduplex with complementary sequences in the RT-PCR DNA fragment comprising the polymorphism; and
e) detecting a real-time decrease in the emission of a signal from the label on the probe,
wherein the decrease in signal indicates the presence of the polymorphism in the RNA target.

21. A kit for the real-time detection of a polymorphism in a target DNA comprising:
a) a pair of amplification primers that can anneal to a target DNA, wherein a first amplification primer anneals upstream of the location of a polymorphism and a second amplification primer anneals downstream of the location of the polymorphism;
b) a probe comprising a detectable label and DNA and RNA nucleic acid sequences, wherein the probe's RNA nucleic acid sequences are entirely complementary to a selected region of the target DNA sequence comprising the polymorphism and the probe's DNA nucleic acid sequences are substantially complementary to DNA sequences adjacent to the selected region of the target DNA sequence;
c) an amplifying polymerase activity, an amplification buffer; and a RNase H activity.

22. The kit of claim 21, wherein the RNA nucleic acid sequence of the probe comprises a sequence that is complimentary to the polymorphism in the target DNA.

23. The kit of claim 21, wherein the polymorphism is a single nucleotide polymorphism (SNP).

24. The kit of claim 21, wherein the DNA and RNA sequences of the probe are covalently linked.

25. The kit of claim 21, wherein the detectable label on the probe is a fluorescent label.

26. The kit of claim 25, wherein the fluorescent label comprises a FRET pair.

27. The kit of claim 21, wherein the probe or PCR fragment is linked to a solid support.

28. A kit for the real-time detection of a polymorphism in a target DNA comprising:
a) a pair of amplification primers that can anneal to a target DNA, wherein a first amplification primer anneals upstream of the location of a polymorphism and a second amplification primer anneals downstream of the location of the polymorphism;
b) a probe comprising a detectable label and DNA and RNA nucleic acid sequences, wherein the probe's RNA nucleic acid sequences are entirely complementary to a selected region of the target DNA sequence comprising the wild type DNA sequence at the location of the polymorphism and the probe's DNA nucleic acid sequences are substantially complementary to DNA sequences adjacent to the selected region of the target DNA sequence;
c) an amplifying polymerase activity, an amplification buffer; and an RNase H activity.

29. A kit for the real-time detection of a polymorphism in a RNA target comprising:
a) a pair of amplification primers that can anneal to a cDNA of the RNA target, wherein a first amplification primer anneals upstream of the location of a polymorphic sequence and a second amplification primer anneals downstream of the location of the polymorphic sequence;
b) a probe comprising a detectable label and DNA and RNA nucleic acid sequences, wherein the probe's RNA nucleic acid sequences are entirely complementary to a selected region of the cDNA comprising the polymorphism and the probe's DNA nucleic acid sequences are substantially complementary to DNA sequences adjacent to the selected region of the cDNA, and
c) a reverse transcriptase activity, an amplifying polymerase activity, reverse transcriptase-PCR buffer; and an RNase H activity.

30. The kit of claim 29, wherein the RNA nucleic acid sequence of the probe comprises a sequence that is complementary to the cDNA at the location of the polymorphism in the target RNA.

31. The kit of claim 29, wherein the polymorphism is a single nucleotide polymorphism (SNP).

32. The kit of claim 29, wherein the DNA and RNA sequences of the probe are covalently linked.

33. The kit of claim 28, wherein the detectable label on the probe is a fluorescent label.

34. The kit of claim 33, wherein the fluorescent label comprises a FRET pair.

35. The kit of claim 29, wherein the probe or reverse transcriptase-PCR fragment is linked to a solid support.

36. The kit of claim 29, wherein the reverse transcriptase activity and the amplifying polymerase activity are found on the same molecule.

37. A kit for the real-time detection of a polymorphism in a RNA target comprising:
a) a pair of amplification primers that can anneal to a cDNA of the RNA target, wherein a first amplification primer anneals upstream of the location of a polymorphic sequence and a second amplification primer anneals downstream of the location of the polymorphic sequence;
b) a probe comprising a detectable label and DNA and RNA nucleic acid sequences, wherein the probe's RNA nucleic acid sequences are entirely complementary to a selected region of the cDNA comprising the wild type DNA sequence at the location of the polymorphism and the probe's DNA nucleic acid sequences are substantially complementary to DNA sequences adjacent to the selected region of the cDNA, and
c) a reverse transcriptase activity, an amplifying polymerase activity, reverse transcriptase-PCR buffer; and an RNase H activity.


SEQUENCE LISTING (33 sequences)

SEQ ID NO: 1 - DNA, 19 bp, Artificial Sequence
  Synthetic oligonucleotide
cgatcaggaaatcaaccag

SEQ ID NO: 2 - DNA, 19 bp, Artificial Sequence
  Synthetic oligonucleotide
cgatcaggcaatcaaccag

SEQ ID NO: 3 - DNA, 27 bp, Artificial Sequence
  Synthetic oligonucleotide
cacactggttgatttcctgatcgcaca

SEQ ID NO: 4 - DNA, 27 bp, Artificial Sequence
  Synthetic oligonucleotide
cacactggttgattgcctgatcgcaca

SEQ ID NO: 5 - DNA, 20 bp, Artificial Sequence
  Synthetic oligonucleotide
tcgtcattccattacctacc

SEQ ID NO: 6 - DNA, 24 bp, Artificial Sequence
  Synthetic oligonucleotide
tactgatcgataatgccagacgaa

SEQ ID NO: 7 - DNA, 18 bp, Artificial Sequence
  Synthetic oligonucleotide
ggcccatccauaacagcc

SEQ ID NO: 8 - DNA, 18 bp, Artificial Sequence
  Synthetic oligonucleotide
ggcccatcccuaacagcc

SEQ ID NO: 9 - DNA, 26 bp, Artificial Sequence
  Synthetic oligonucleotide
gagaggctgttatggatgggccgaga

SEQ ID NO: 10 - DNA, 26 bp, Artificial Sequence
  Synthetic oligonucleotide
gagaggctgttagggatgggccgaga

SEQ ID NO: 11 - DNA, 25 bp, Artificial Sequence
  Synthetic oligonucleotide
gatgaactccaggataaaatccacc

SEQ ID NO: 12 - DNA, 21 bp, Artificial Sequence
  Synthetic oligonucleotide
tacttcaggctgaaggaaagg

SEQ ID NO: 13 - protein, 224 aa, Pyrococcus furiosus
MKIGGIDEAGRGPAIGPLVVATVVVDEKNIEKLRNIGVKDSKQLTPHERKNLFSQITSIADDYKIVIVSPEEIDNRSGTMNELEVEKFALALNSLQIKPALIYADAADVDANRFASLIERRLNYKAKIIAEHKADAKYPVVSAASILAKVVRDEEIEKLKKQYGDFGSGYPSDPKTKKWLEEYYKKHNSFPPIVRRTWETVRKIEESIKAKKSQLTLDKFFKKP

SEQ ID NO: 14 - protein, 220 aa, Pyrococcus horikoshi
MKVAGVDEAGRGPVIGPLVIGVAVIDEKNIERLRDIGVKDSKQLTPGQREKLFSKLIDILDDYYVLLVTPKEIDERHHSMNELEAEKFVVALNSLRIKPQKIYVDSADVDPKRFASLIKAGLKYEATVIAEHKADAKYEIVSAASIIAKVTRDREIEKLKQKYGEFGSGYPSDPRTKEWLEEYYKQYGDFPPIVRRTWETARKIEERFRKNQLTLDKFLK

SEQ ID NO: 15 - protein, 228 aa, Thermococcus kodakarensis
MKIAGIDEAGRGPVIGPMVIAAVVVDENSLPKLEELKVRDSKKLTPKRREKLFNEILGVLDDYVILELPPDVIGSREGTLNEFEVENFAKALNSLKVKPDVIYADAADVDEERFARELGERLNFEAEVVAKHKADDIFPVVSAASILAKVTRDRAVEKLKEEYGEIGSGYPSDPRTRAFLENYYREHGEFPPIVRKGWKTLKKIAEKVESEKKAEERQATLDRYFRKV

SEQ ID NO: 16 - protein, 211 aa, Archaeoglobus profundus
MIAGIDEAGKGPVIGPLVICGVLCDEETVEYLKSVGVKDSKKLDRRKREELYNIIKSLCKVKVLKISVEDLNRLMEYMSINEILKRAYVEIIRSLMPKVVYIDCPDINVERFKHEIEERTGVEVFASHKADEIYPIVSIASIVAKVERDFEIDKLKKIYGDFGSGYPSDLRTIEFLRSYLREHKSFPPIVRKRWKTLKRLTTHTLSDFFEV

SEQ ID NO: 17 - protein, 205 aa, Archaeoglobus fulgidis
MKAGIDEAGKGCVIGPLVVAGVACSDEDRLRKLGVKDSKKLSQGRREELAEEIRKICRTEVLKVSPENLDERMAAKTINEILKECYAEIILRLKPEIAYVDSPDVIPERLSRELEEITGLRVVAEHKADEKYPLVAAASIIAKVEREREIERLKEKFGDFGSGYASDPRTREVLKEWIASGRIPSCVRMRWKTVSNLRQKTLDDF

SEQ ID NO: 18 - protein, 233 aa, Thermococcus celer
LKLAGIDEAGRGPVIGPMVIAAVVLDEKNVPKLRDLGVRDSKKLTPKRRERLFNDIIKLLDDYVILELWPEEIDSRGGTLNELEVERFVEALNSLKVKPDVVYIDAADVKEGRFGEEIKERLNFEAKIVSEHRADDKFLPVSSASILAKVTRDRAIEKLKEKYGEIGSGYPSDPRTREFLENYYRQHGEFPPVVRRSWKTLRKIEEKLRKEAGSKNPENSKEKGQTSLDVFLR

SEQ ID NO: 19 - protein, 224 aa, Thermococcus litoralis
MKLGGIDEAGRGPVIGPLVIAAVVVDESRMQELEALGVKDSKKLTPKRREELFEEIVQIVDDHVIIQLSPEEIDGRDGTMNELEIENFAKALNSLKVKPDVLYIDAADVKEKRFGDIIGERLSFSPKIIAEHKADSKYIPVAAASILAKVTRDRAIEKLKELYGEIGSGYPSDPNTRRFLEEYYKAHGEFPPIVRKSWKTLRKIEEKLKAKKTQPTILDFLKKP

SEQ ID NO: 20 - protein, 16 aa, Pyrococcus furiosis
GIDEAGRGPAIGPLVV

SEQ ID NO: 21 - protein, 12 aa, Pyrococcus furiosis
LRNIGVKDSKQL

SEQ ID NO: 22 - protein, 19 aa, Pyrococcus furiosis
HKADAKYPVVSAASILAKV

SEQ ID NO: 23 - protein, 16 aa, Pyrococcus furiosis
KLKKQYGDFGSGYPSD

SEQ ID NO: 24 - protein, 174 aa, Haemophilus influenzae
MFNLSLSIKIPAILHNNLFVMQKQIEIFTDGSCLGNPGAGGIGAVLRYKQHEKMLSKGYFKTTNNRMELRAVIEALNTLKEPCLITLYSDSQYMKNGITKWIFNWKKNNWKASSGKPVKNQDLWIALDESIQRHKINWQWVKGHAGHRENEICDELAKKGAENPTLEDMGYFEE

SEQ ID NO: 25 - protein, 166 aa, Thermus thermophilus
MNPSPRKRVALFTDGACLGNPGPGGWAALLRFHAHEKLLSGGEACTTNNRMELKAAIEGLKALKEPCEVDLYTDSHYLKKAFTEGWLEGWRKRGWRTAEGKPVKNRDLWEALLLAMAPHRVRFHFVKGHTGHPENERVDREARRQAQSQAKTPCPPRAPTLFHEEA

SEQ ID NO: 26 - protein, 161 aa, Thermus aquaticus
MSLPLKRVDLFTDGACLGNPGPGGWAALLRYGSQEKLLSGGEPCTTNNRMELRAALEGLLALREPCQVHLHTDSQYLKRAFAEGWVERWQRNGWRTAEGKPVKNQDLWQALLKAMEGHEVAFHFVEGHSGHPENERVDREARRQAKAQPQVPCPPKEATLF

SEQ ID NO: 27 - protein, 146 aa, Salmonella enterica
MLKQVEIFTDGSCLGNPGPGGYGAILRYRGHEKTFSEGYTLTTNNRMELMAAIVALEALKEHCEVTLSTDSQYVRQGITQWIHNWKKRGWKTAEKKPVKNVDLWKRLDAALGQHQIKWVWVKGHAGHPENERCDELARAAAMNPTQ

SEQ ID NO: 28 - protein, 146 aa, Agrobacterium tumefaciens
MKHVDIFTDGACSGNPGPGGWGAVLRYGETEKELSGGEADTTNNRMELLAAISALNALKSPCEVDLYTDSAYVKDGITKWIFGWKKKGWKTADNKPVKNVELWQALEAAQERHKVTLHWVKGHAGHPENERADELARKGMEPFKRR

SEQ ID NO: 29 - protein, 25 aa, Artificial Sequence
  Synthetic polypeptide
KXVXLFTDGXCXGXPGXGGXALLRY

SEQ ID NO: 30 - protein, 8 aa, Artificial Sequence
  Synthetic polypeptide
TTNNRMEL

SEQ ID NO: 31 - protein, 5 aa, Artificial Sequence
  Synthetic polypeptide
KPVKN

SEQ ID NO: 32 - protein, 12 aa, Artificial Sequence
  Synthetic polypeptide
FVKGHXGHXENE

SEQ ID NO: 33 - DNA, 1950 bp, Salmonella enterica
aacagtgctcgtttacgacctgaattactgattctggtactaatggtgatgatcatttctatgttcgtcattccattacctacctatctggttgatttcctgatcgcactgaatatcgtactggcgatattggtgtttatggggtcgttctacattgacagaatcctcagtttttcaacgtttcctgcggtactgttaattaccacgctctttcgtctggcattatcgatcagtaccagccgtcttatcttgattgaagccgatgccggtgaaattatcgccacgttcgggcaattcgttattggcgatagcctggcggtgggttttgttgtcttctctattgtcaccgtggtccagtttatcgttattaccaaaggttcagaacgcgtcgcggaagtcgcggcccgattttctctggatggtatgcccggtaaacagatgagtattgatgccgatttgaaggccggtattattgatgcggatgctgcgcgcgaacggcgaagcgtactggaaagggaaagccagctttacggttcctttgacggtgcgatgaagtttatcaaaggtgacgctattgccggcatcattattatctttgtgaactttattggcggtatttcggtggggatgacccgccatggtatggatttgtcctccgctctgtctacttataccatgctgaccattggtgatggtcttgtcgcccagatccccgcattgttgattgcgattagtgccggttttatcgtgactcgcgtaaatggcgatagcgataatatggggcggaatatcatgacgcagctgttgaacaacccatttgtattggttgttacggctattttgaccatttcaatgggaactctgccgggattcccgctgccggtatttgttattttatcggtggttttaagcgtactcttctattttaaattccgtgaagcaaaacgtagcgccgccaaacctaaaaccagcaaaggcgagcagccgcttagtattgaggaaaaagaagggtcgtcgttgggactgattggcgatctcgataaagtctctacagagaccgtaccgttgatattacttgtgccgaagagccggcgtgaagatctggaaaaagctcaacttgcggagcgtctacgtagtcagttctttattgattatggcgtgcgcctgccggaagtattgttacgcgatggcgagggcctggacgataacagcatcgtattgttgattaatgagatccgtgttgaacaatttacggtctattttgatttgatgcgagtggtaaattattccgatgaagtcgtgtcctttggtattaatccaacaatccatcagcaaggtagcagtcagtatttctgggtaacgcatgaagagggggagaaactccgggagcttggctatgtgttgcggaacgcgcttgatgagctttaccactgtctggcggtgaccgtggcgcgcaacgtcaatgaatatttcggtattcaggaaacaaaacatatgctggaccaactggaagcgaaatttcctgatttacttaaagaagtgctcagacatgccacggtacaacgtatatctgaagttttgcagcgtttattaagcgaacgtgtttccgtgcgtaatatgaaattaattatggaagcgctcgcattgtgggcgccaagagaaaaagatgtcattaaccttgtagagcatattcgtggagcaatggcgcgttatatttgtcataaattcgccaatggcggcgaattacgagcagtaatggtatctgctgaagttgaggatgttattcgcaaagggatccgtcagacctctggcagtaccttcctcagccttgacccggaagcctccgctaatttgatggatctcattacacttaagttggatgatttattgattgcacataaagatcttgtcctccttacgtctgtcgatgtccgtcgatttattaagaaa